(19) United States
(12) Patent Application Publication
(10) Pub. No.: US 2024/0287583 A1
(43) Pub. Date: Aug. 29, 2024

(54) APTAMER DYNAMIC RANGE COMPRESSION AND DETECTION TECHNIQUES

(71) Applicant: ILLUMINA, INC., San Diego, CA (US)

(72) Inventors: Andrew Slatter, Cambridge (GB); Carlo Randise-Hinchliff, San Diego, CA (US); Andrew Price, San Mateo, CA (US); Niall Anthony Gormley, Little Chesterfield (GB); Andrea Manzo, San Diego, CA (US); Nithya Subramanian, Cambridge (GB); Fiona Kaper, Solana Beach, CA (US); David Jones, Huntingdon (GB); Steven Norberg, La Mesa, CA (US)

(21) Appl. No.: 18/571,069

(22) PCT Filed: Apr. 6, 2023
(86) PCT No.: PCT/US2023/017778

§ 371 Date: Dec. 15, 2023

Related U.S. Application Data
(60) Provisional application No. 63/329,101, filed on Apr. 8, 2022.
(60) Provisional application No. 63/343,760, filed on May 19, 2022.
(60) Provisional application No. 63/347,375, filed on May 31, 2022.
(60) Provisional application No. 63/385,544, filed on Nov. 30, 2022.

(51) Int. Cl. C12Q 1/682; C12Q 1/6839

(52) U.S. Cl. CPC C12Q 1/682; C12Q 1/6839

(57) ABSTRACT

Aptamer detection techniques with dynamic range compression are described that permit removal of a portion of more abundant aptamers in an aptamer-based assay. In an embodiment, a mixture of tagged probes and dummy probes can be used such that the dummy probes bind abundant aptamers and in turn are not captured or amplified for detection in downstream steps. Other techniques are also contemplated, including targeted removal of or cleavage of probes that bind to excess aptamers.

### CROSS-REFERENCE TO RELATED APPLICATIONS

The present application is a 371 national stage application of PCT/US2023/017778, filed on Apr. 6, 2023, which claims priority to and the benefit of U.S. Provisional Application No. 63/329,101 filed Apr. 8, 2022. The present application also claims priority to and the benefit of U.S. Provisional Application No. 63/343,760 filed May 19, 2022. The present application also claims priority to and the benefit of U.S. Provisional Application No. 63/347,375 filed May 31, 2022. The present application also claims priority to and the benefit of U.S. Provisional Application No. 63/385,544 filed Nov. 30, 2022. The disclosures of all of these are hereby incorporated by reference in their entireties herein.

### REFERENCE TO ELECTRONIC SEQUENCE LISTING

The application contains a Sequence Listing which has been submitted electronically in XML format and is hereby incorporated by reference in its entirety. Said .XML copy, created on Apr. 5, 2023, is named “IP-2487-PCT.xml” and is 19,152 bytes in size. The sequence listing contained in this .XML file is part of the specification and is hereby incorporated by reference herein in its entirety

### BACKGROUND

The disclosed technology relates generally to aptamer detection and/or identification techniques for dynamic range compression in conjunction with an aptamer-based assay. In particular, the technology disclosed relates to nucleic acid sequencing for direct or indirect aptamer detection in conjunction with an aptamer-based assay.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Protein expression patterns help define a cell's identity and state. RNA transcripts are often used as a surrogate for protein expression, but the relationship between abundance of proteins and mRNA is not one-to-one. There are differences caused by regulation of posttranscriptional, translational and protein degradation. Therefore, direct nucleic acid sequencing of RNA transcripts may not provide an accurate estimation of protein expression.

Aptamers are nucleic acids that bind to molecular targets, such as proteins, with high affinity and specificity. Advancements in aptamer selection and design include Systematic Evolution of Ligands by EXponential enrichment (SELEX). In SELEX, high affinity nucleic acids for different analytes of interest can be isolated from a combinatorial library, permitting high throughput characterization of aptamer-target binding and multiplexed assays for analytes in a complex biological sample. Upon aptamer binding to an analyte target, the binding event can be detected to characterize the presence and concentration of various analytes in the biological sample. However, because protein or other analyte concentrations can vary to a high degree within and/or between different biological samples, identifying a useful detection range for a multiplexed aptamer-based assay is difficult.

### BRIEF DESCRIPTION

In one embodiment, the present disclosure provides method of aptamer detection that includes contacting analytes of a sample with a plurality of aptamers under conditions that permit analyte-aptamer complexes to form, wherein different aptamers of the plurality of aptamers have specific affinity for respective different analytes of the analytes. The method also includes detecting the analytes by detecting aptamers of the analyte-aptamer complexes. Detecting an individual aptamer of the plurality of aptamers includes contacting the individual aptamer with a mixture of first probes, wherein a first complementary region of each first probe of the mixture hybridizes to a first region of the individual aptamer and wherein only a subset of the first probes in the mixture are coupled to an affinity tag. The detecting also includes contacting the individual aptamer with a second probe to hybridize a second complementary region of the second probe to a second region of the individual aptamer and wherein the second probe comprises a nonhybridizing region extending from the complementary region, the nonhybridizing region comprising an identification sequence uniquely identifying for the individual aptamer, wherein the first complementary region and the second complementary region uniquely hybridize to the individual aptamer. The detecting also includes capturing a first probe of the mixture via binding of the affinity tag to an affinity tag binder to capture the individual aptamer and the second probe hybridized to the second region of the individual aptamer, wherein the first probe is in the subset coupled to the affinity tag. The detecting also includes detecting the identification sequence of the captured second probe

In one embodiment, the present disclosure provides an aptamer detection probe set. The aptamer detection probe set includes a plurality of different first probe mixtures complementary to respective different aptamers of an aptamer panel. An individual first probe mixture of the plurality of different first probe mixtures includes a binding subset of first probes coupled to an affinity tag; and dummy subset of first probes, and wherein each probe in the binding subset and the dummy subset of the individual first probe mixture comprises a same binding region that is complementary to a first sequence of an individual aptamer of the aptamer panel. The aptamer detection probe set also includes a plurality of different second probes complementary to the respective different aptamers of the aptamer panel, wherein an individual second probe of the plurality of different second probes comprises a second binding region complementary to a second sequence of the individual aptamer and wherein the individual second probe comprises a nonhybridizing region, the nonhybridizing region comprising an identification sequence uniquely identifying for the individual aptamer. In certain embodiments, the aptamer detection probe set can be used to generate oligonucleotides suitable for next generation sequencing techniques. Thus, aptamer detection using the aptamer detection probe set can include sequencing of the identification sequence in the generated oligonucleotides using next generation sequencing (NGS) techniques. Further, the generated oligonucleotides can include one or more adapter sequences and/or sample-specific index sequences to allow for multiplexing in a single sequencing run.

In one embodiment, the present disclosure provides an aptamer detection probe set. The aptamer detection probe set includes a plurality of different first probe mixtures complementary to respective different aptamers of an aptamer panel. Each first probe mixture of the plurality of different first probe mixtures includes a binding subset of first probes; and a dummy subset of first probes not coupled to affinity tag, and wherein each first probe mixture comprises a binding region that is complementary to an aptamer of the aptamer panel, and wherein the binding region is unique to each first probe mixture such that each first probe mixture binds to a different aptamer of the aptamer panel, wherein each first probe mixture has a different ratio of the binding subset to the dummy subset relative to the other first probe mixtures of the plurality. The aptamer detection probe set also includes a plurality of different second probes complementary to the respective different aptamers of the aptamer panel.

In one embodiment, the present disclosure provides an aptamer detection probe set. The aptamer detection probe set includes a plurality of different reporter probe mixtures complementary to respective different aptamers of an aptamer panel. An individual reporter probe mixture of the plurality of different reporter probe mixtures includes a first subset of reporter probes comprising an amplifiable nonhybridizing region, the nonhybridizing region comprising an identification sequence uniquely identifying for the individual aptamer that is flanked by a first primer region and a second primer region and that is capable of being amplified using primers complementary to or corresponding to (e.g., having a same sequence as) the first primer region and the second primer region; and a second subset of reporter probes, and wherein each probe in the first subset and the second subset of the individual reporter probe mixture comprises a same binding region that is complementary to a first sequence of an individual aptamer of the aptamer panel, wherein the individual second probe comprises a nonamplifiable nonhybridizing region that is not capable of being amplified using the primers. The aptamer detection probe set also includes a plurality of different capture probes complementary to the respective different aptamers of the aptamer panel, wherein an individual capture probe of the plurality of different capture probes comprises a second binding region complementary to a second sequence of the individual aptamer and, wherein each capture probe of the plurality is coupled to an affinity tag.

In one embodiment, the present disclosure provides a method of aptamer detection that includes contacting an individual aptamer with reporter probes that hybridize a first region of the individual aptamer, wherein a first subset of the reporter probes comprise an amplifiable nonhybridizing region, the amplifiable nonhybridizing region comprising an identification sequence uniquely identifying for the individual aptamer that is flanked by a first primer region and a second primer region and a second subset comprise a nonamplifiable nonhybridizing region; contacting the individual aptamer with a capture probe, wherein the capture probes hybridize to a second region of the individual aptamer and wherein the capture probe is associated with an affinity tag; capturing the capture probe via binding of the affinity tag to an affinity tag binder to capture the individual aptamer and the reporter probe comprising the amplifiable nonhybridizing region hybridized to the first region of the individual aptamer; and detecting the identification sequence of the captured reporter probe via amplification of the identification sequence.

In one embodiment, the present disclosure provides a method of aptamer detection that includes contacting aptamers with probes that hybridize to respective different aptamers, wherein a complementary region of each probe hybridizes to an individual aptamer of the aptamers and wherein each probe comprises a nonhybridizing region extending from the complementary region, the nonhybridizing region comprising an identification sequence uniquely identifying for each individual aptamer; using an exonuclease to remove excess probes not hybridized to the aptamers; and detecting the identification sequence in the captured probes after removing the excess probes.

In one embodiment, the present disclosure provides a method of aptamer detection that includes contacting a first aptamer and a second aptamer with reporter probes, the reporter probes comprising a first subset that hybridize to the first aptamer and a second subset that hybridize to the second aptamer, wherein the reporter probes of the first subset comprise a first nonhybridizing region comprising a first identification sequence uniquely identifying for the first aptamer and a first group capture sequence and wherein the reporter probes of the second subset comprise a second nonhybridizing region comprising a second identification sequence uniquely identifying for the second aptamer and the first group capture sequence. The method also includes contacting the first aptamer and the second aptamer with capture probes, wherein the capture probes hybridize to the first aptamer or the second aptamer and wherein the capture probes are associated with an affinity tag; capturing the capture probes via binding of the affinity tag to an affinity tag binder to capture the first aptamer and the second aptamer and the reporter probes; generating first oligonucleotides comprising the first identification sequence and the first group capture sequence from the first subset and second oligonucleotides comprising the second identification sequence and the first group capture sequence from the second subset; capturing the first oligonucleotides and the second oligonucleotides using a first group of beads carrying a sequence complementary to the first group capture sequence; and detecting the first identification sequence in the captured first oligonucleotides and the second identification sequence in the second oligonucleotides to detect the first aptamer and the second aptamer.

In one embodiment, the present disclosure provides a method of aptamer detection that includes contacting an individual aptamer with a first reporter probe that hybridizes to a first region of the individual aptamer, wherein the first reporter probe comprises a first nonhybridizing region, the nonhybridizing region comprising a first identification sequence uniquely identifying for the individual aptamer and with a second reporter probe that hybridize to a second region of the individual aptamer, wherein the second reporter probe comprises a second nonhybridizing region, the second nonhybridizing region comprising a second identification sequence uniquely identifying for the individual aptamer; ligating ends of the first identification sequence and the second identification sequence to one another to generate ligated reporter probes; capturing ligated reporter probes using an affinity tag coupled to the first reporter probe or the second reporter probe; and detecting the first identification sequence and the second identification sequence via amplification of the captured ligated reporter probes to detect the individual aptamer.

In one embodiment, the present disclosure provides a method of aptamer detection that includes contacting analytes of a sample with a plurality of aptamers under conditions that permit analyte-aptamer complexes to form, wherein different aptamers of the plurality of aptamers have specific affinity for respective different analytes of the analytes; detecting the analytes by detecting aptamers of the analyte-aptamer complexes, wherein detecting an individual aptamer of the plurality of aptamers comprises: contacting the individual aptamer with a mixture of first probes, wherein a first complementary region of each first probe of the mixture is capable of hybridizing to a first region of the individual aptamer and wherein only a subset of the first probes in the mixture are coupled to an affinity tag such that a first probe of the mixture hybridizes to the first region of the individual aptamer; contacting the individual aptamer with a second probe to hybridize a second complementary region of the second probe to a second region of the individual aptamer and wherein the second probe comprises a nonhybridizing region extending from the complementary region, the nonhybridizing region comprising an identification sequence uniquely identifying for the individual aptamer, wherein the first complementary region and the second complementary region uniquely hybridize to the individual aptamer; ligating the first probe hybridized to the first region of the individual aptamer to the second probe hybridized to the second region of the individual aptamer; capturing the first probe via binding of the affinity tag to an affinity tag binder to capture the individual aptamer and the second probe hybridized to the second region of the individual aptamer and ligated to the first probe, wherein the first probe is in the subset coupled to the affinity tag; and detecting the identification sequence of the captured second probe.

In one embodiment, the present disclosure provides a method of aptamer detection that includes contacting analytes of a sample with a plurality of aptamers under conditions that permit analyte-aptamer complexes to form, wherein different aptamers of the plurality of aptamers have specific affinity for respective different analytes of the analytes. The method also includes detecting the analytes by detecting aptamers of the analyte-aptamer complexes. The detecting includes contacting the individual aptamer with a mixture of first probes, wherein a first complementary region of each first probe of the mixture hybridizes to a first region of the individual aptamer and wherein only a subset of the first probes in the mixture are coupled to an affinity tag; contacting the individual aptamer with a second probe to hybridize a second complementary region of the second probe to a second region of the individual aptamer and wherein the second probe comprises a nonhybridizing region extending from the complementary region, the nonhybridizing region comprising an identification sequence uniquely identifying for the individual aptamer, wherein the first complementary region and the second complementary region uniquely hybridize to the individual aptamer; capturing a first probe of the mixture via binding of the affinity tag to an affinity tag binder to capture the individual aptamer and the second probe hybridized to the second region of the individual aptamer, wherein the first probe is in the subset coupled to the affinity tag; generating amplification products from the captured second probe using a primer pair, wherein the primer pair comprises a first primer complementary to a region of the second probe that does not include the second complementary region and that does not include the identification sequence; and sequencing the amplification products.

In one embodiment, the present disclosure provides a method of sequencing that includes generating sequence data from a sequence library. The sequence library is prepared by contacting analytes of a sample with a plurality of aptamers under conditions that permit analyte-aptamer complexes to form, wherein different aptamers of the plurality of aptamers have specific affinity for respective different analytes of the analytes; forming a first plurality of aptamer complexes of a first type by hybridizing a reporter probe and a dummy probe to an individual aptamer of the plurality of aptamers, wherein the dummy probe comprises a first complementary region that hybridizes to a first region of the individual aptamer and wherein the reporter probe comprises a second complementary region that hybridizes to a second region of the individual aptamer and a nonhybridizing region extending from the complementary region, the nonhybridizing region comprising an identification sequence uniquely identifying for the individual aptamer; forming a second plurality of aptamer complexes of a second type by hybridizing the reporter probe and a capture probe to the individual aptamer, wherein the capture probe comprises the first complementary region that hybridizes to a first region of the individual aptamer and an affinity tag; the second plurality of aptamer complexes from the first plurality via the affinity tag to generate a separated second plurality of aptamer complexes; and amplifying a portion of the reporter probes of the separated second plurality of aptamer complexes to generate the sequence library. The method also includes identifying the identification sequence in the sequence data; and generating a notification that the individual aptamer is present in the sample based on the identifying. In embodiments, the method also includes quantifying the identified identification sequence in the sequence data to measure relative amounts of aptamer present in the sample. The number of the identified identification sequences allows for the relative amounts of the aptamer to be quantified.

### BRIEF DESCRIPTION OF THE DRAWINGS

These and other features, aspects, and advantages of the disclosed embodiments will become better understood when the following detailed description is read with reference to the accompanying drawings in which like characters represent like parts throughout the drawings, wherein:

FIG. 1 is a schematic illustration of an example dynamic range within a sample, according to an embodiment;

FIG. 2 shows an example workflow for dynamic range compression, according to an embodiment;

FIG. 3 shows an example workflow for dynamic range compression using different probe mixes based on aptamer abundancy, according to an embodiment;

FIG. 4 is a schematic illustration of capture probe and reporter probe separation, according to an embodiment;

FIG. 5 is a schematic illustration of a tri-molecular complex for use in conjunction with the dynamic range compression techniques, according to an embodiment;

FIG. 6 shows example arrangements of a nonhybridizing region, according to an embodiment;

FIG. 7 shows example reporter probe direct amplification techniques, according to an embodiment;

FIG. 8A shows example sequencing from direct amplification techniques, according to an embodiment;

FIG. 8B shows results of sequencing using the technique of Option 1 of FIG. 8A;

FIG. 8C shows sequencing quality metrics of the sequencing reaction of FIG. 8B;

FIG. 9A shows example reporter probe step out amplification techniques, according to an embodiment;

FIG. 9B shows results of amplification using the technique of Option 1 of FIG. 9A;

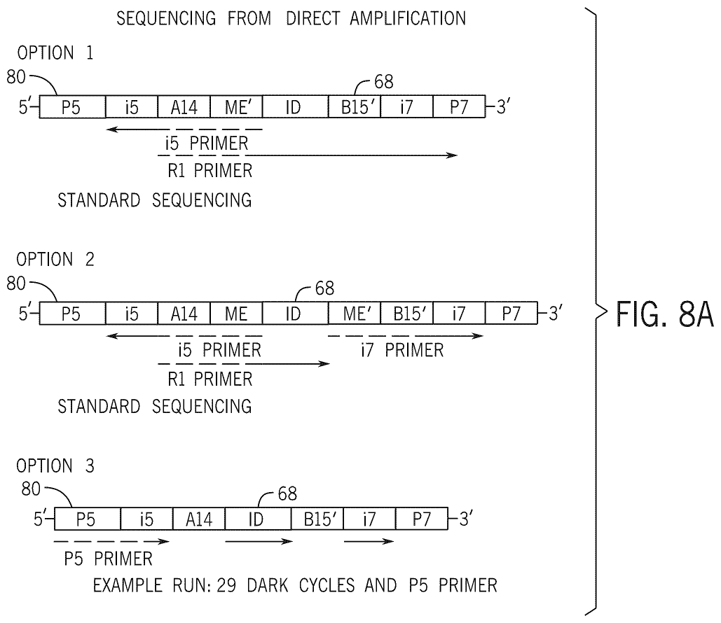

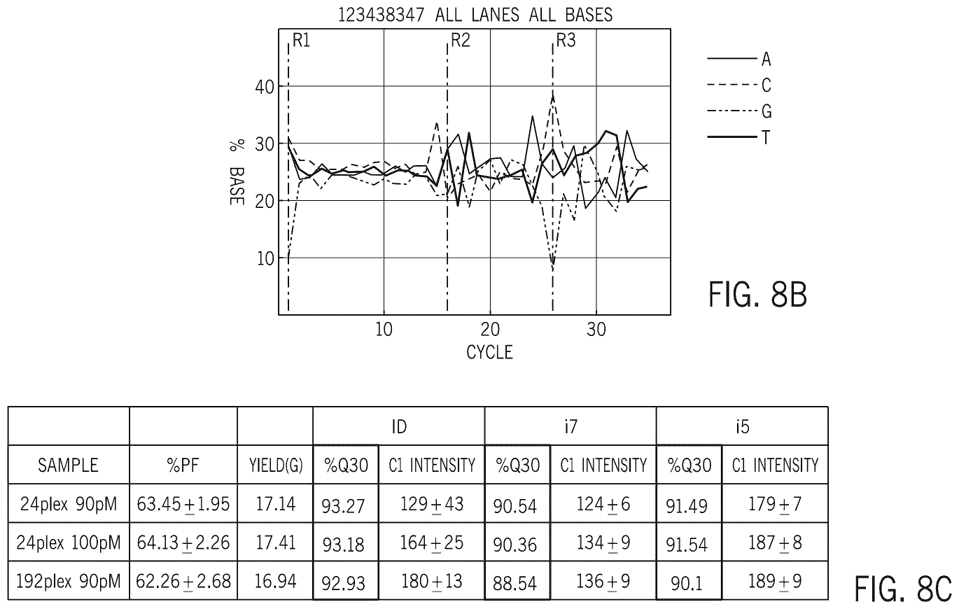

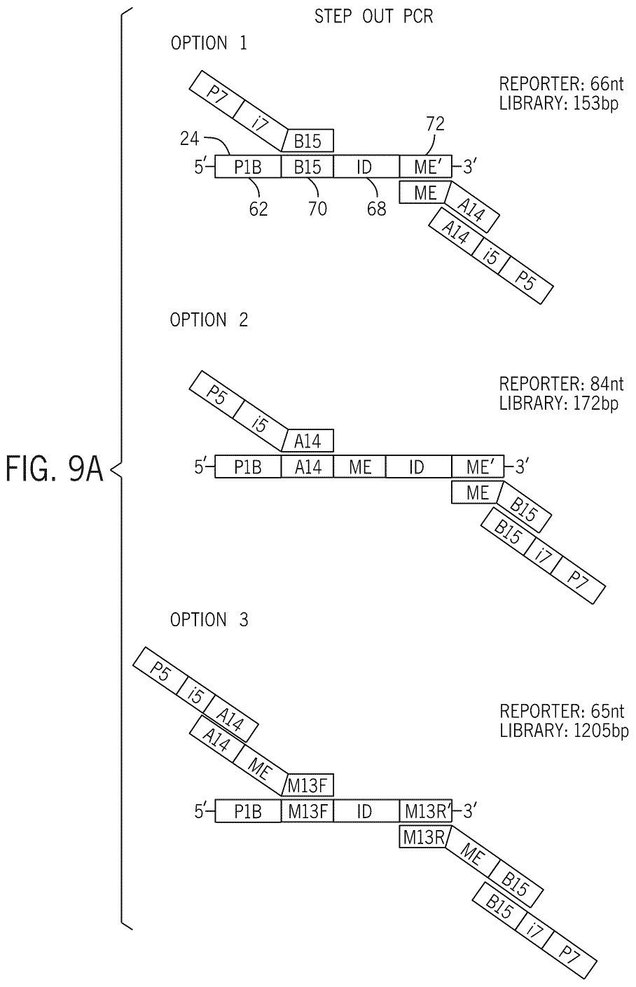

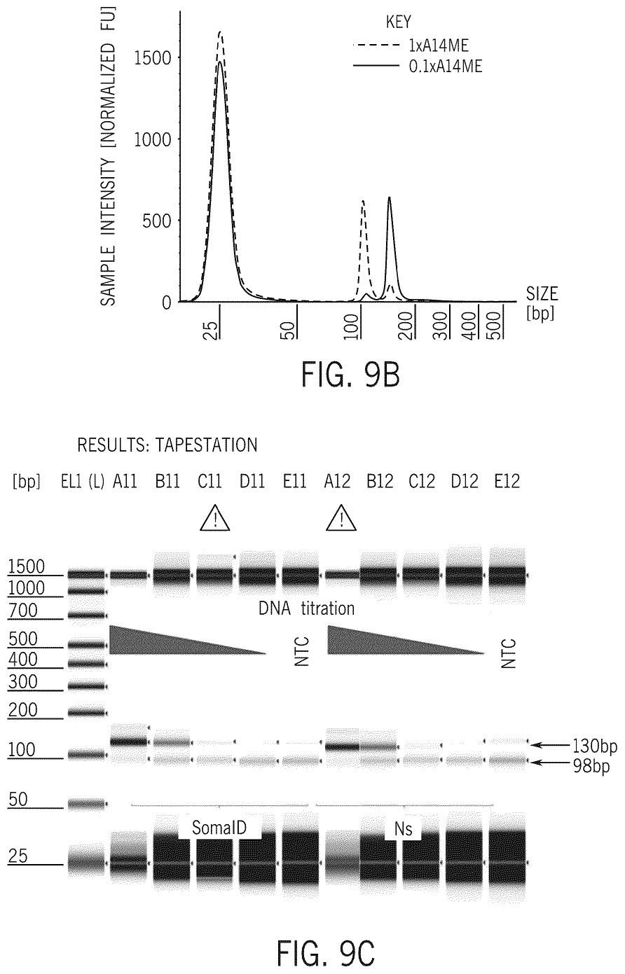

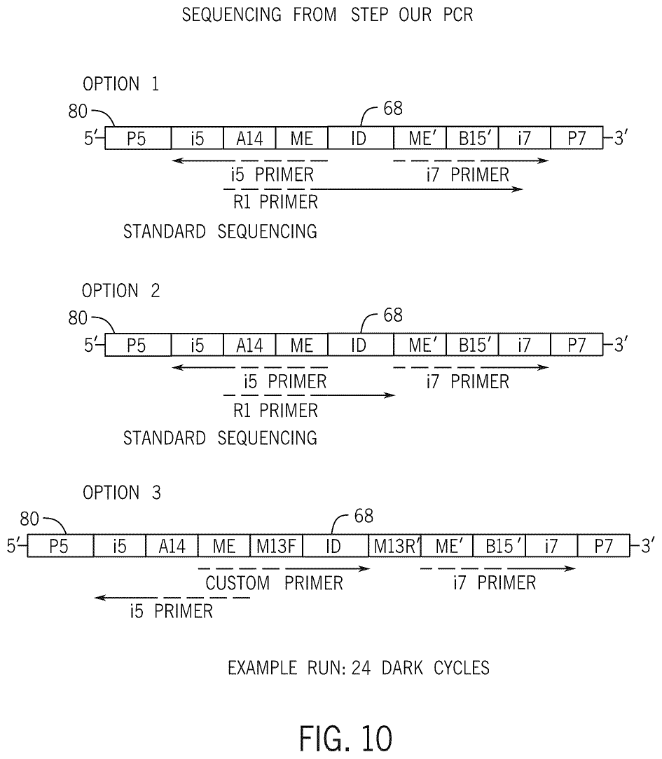

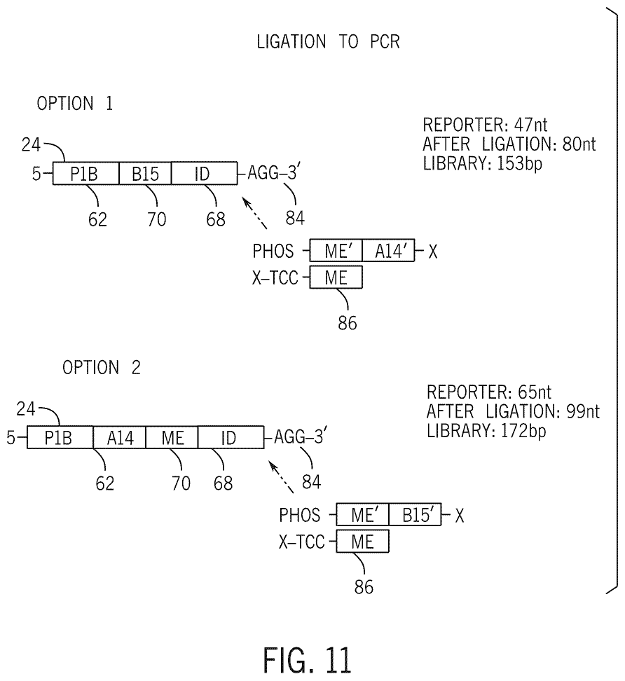

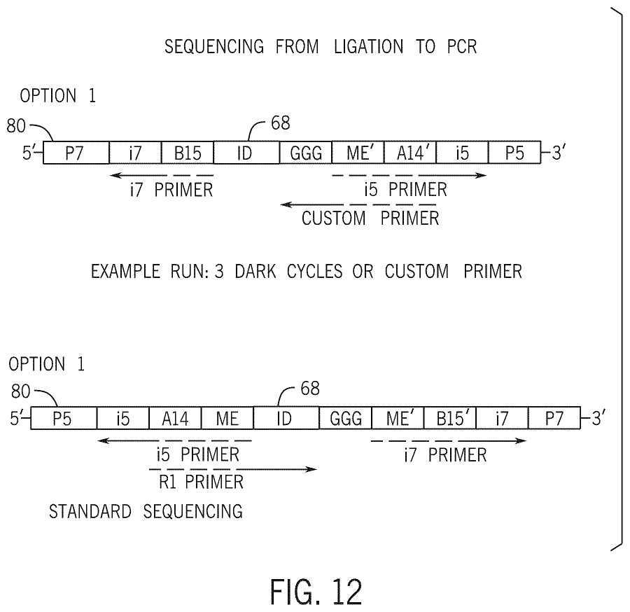

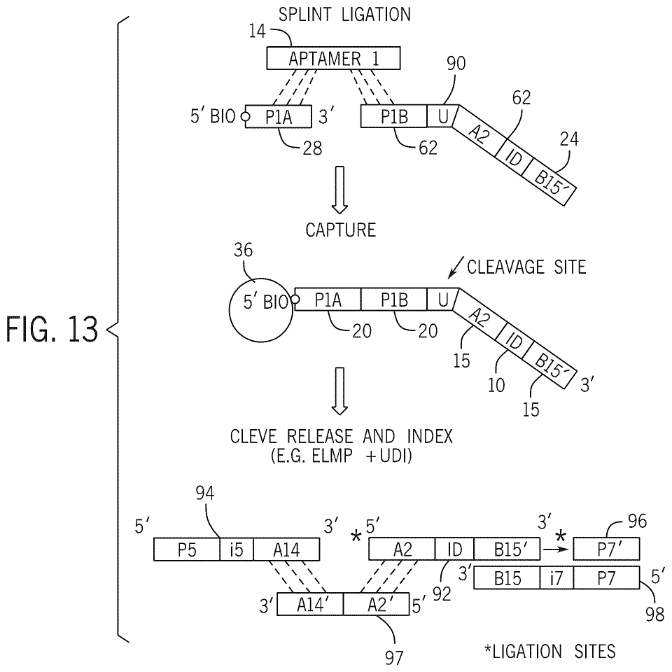

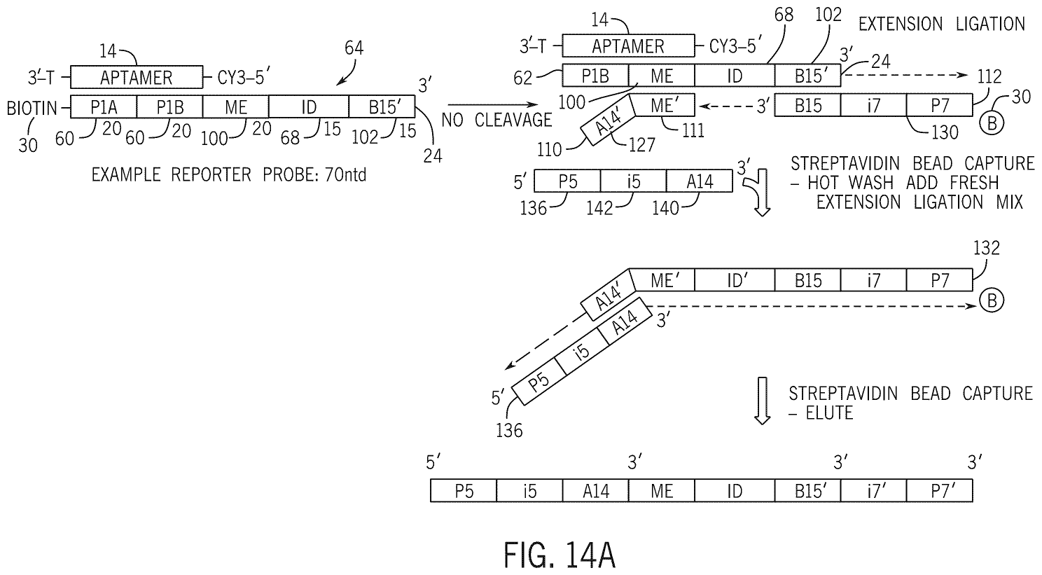

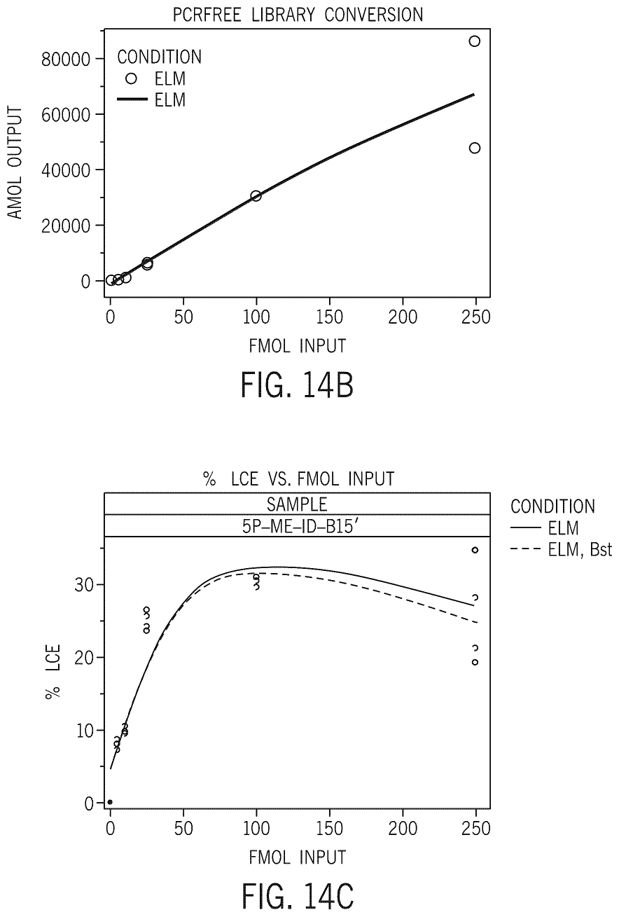

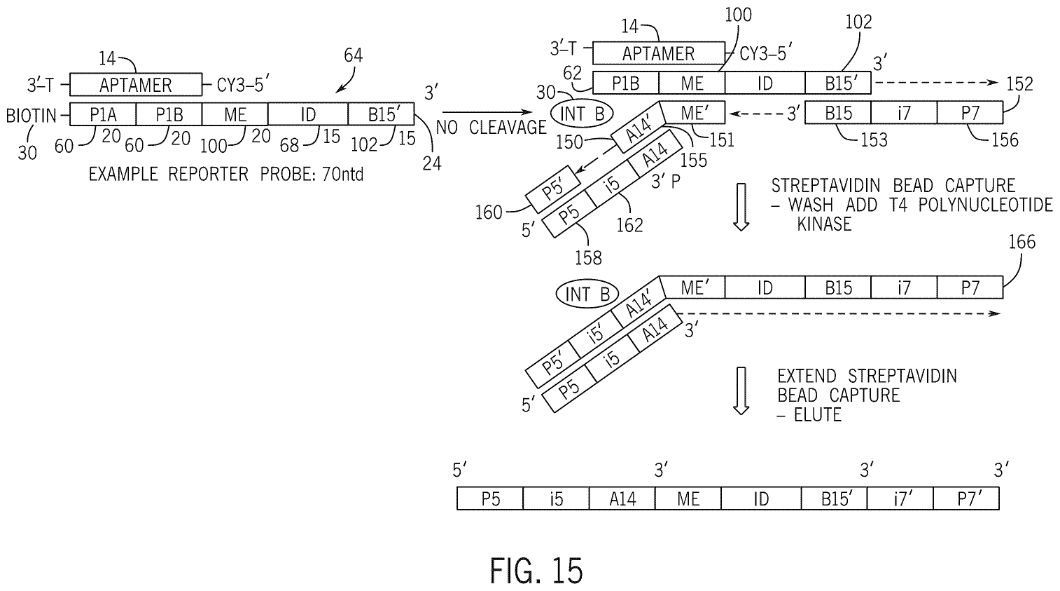

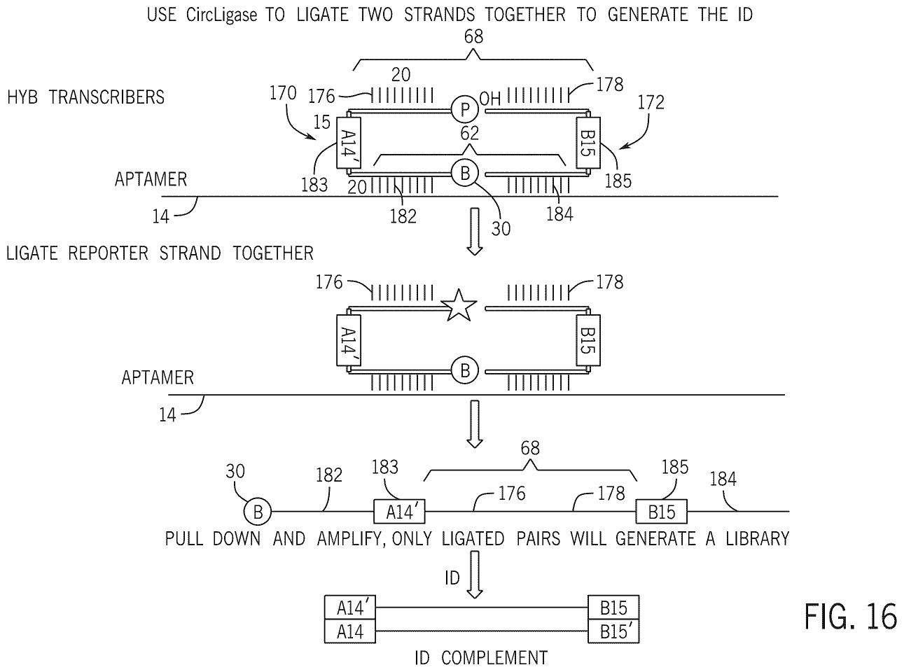

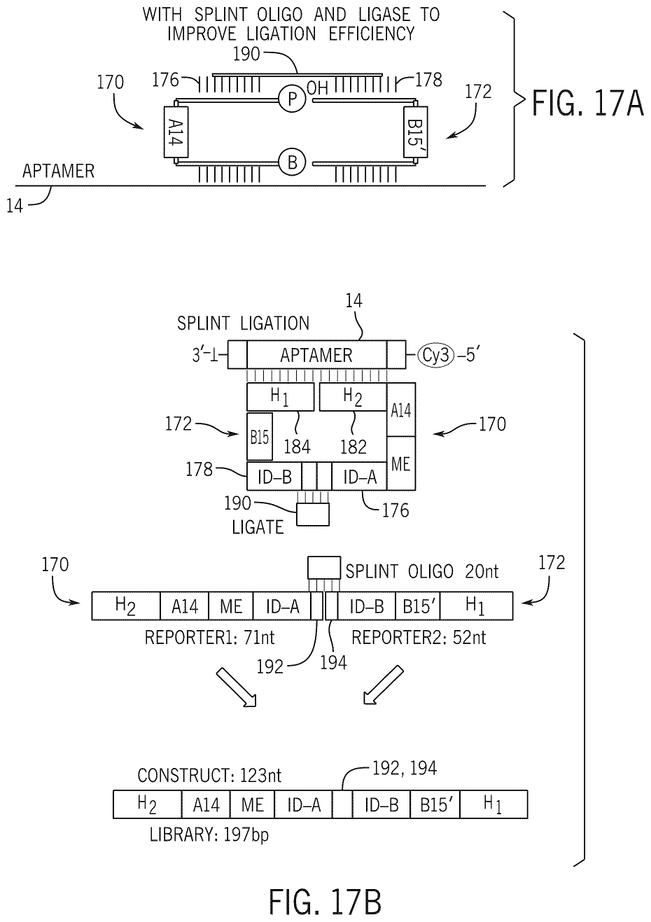

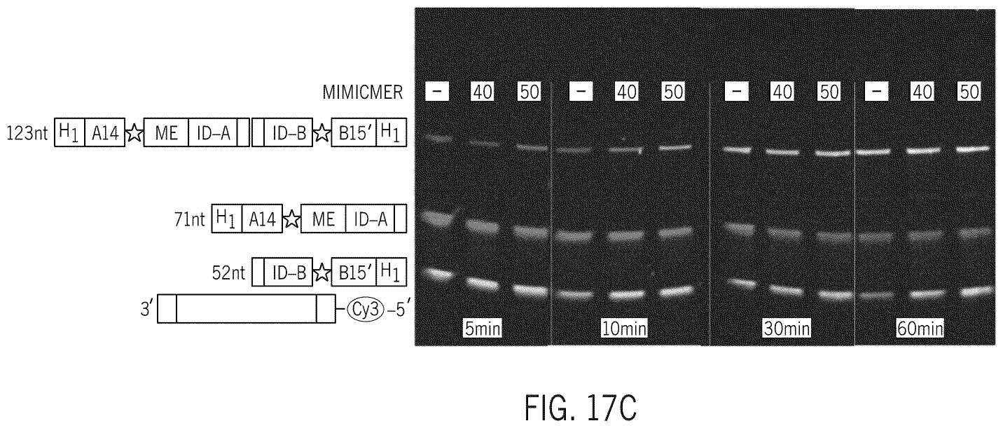

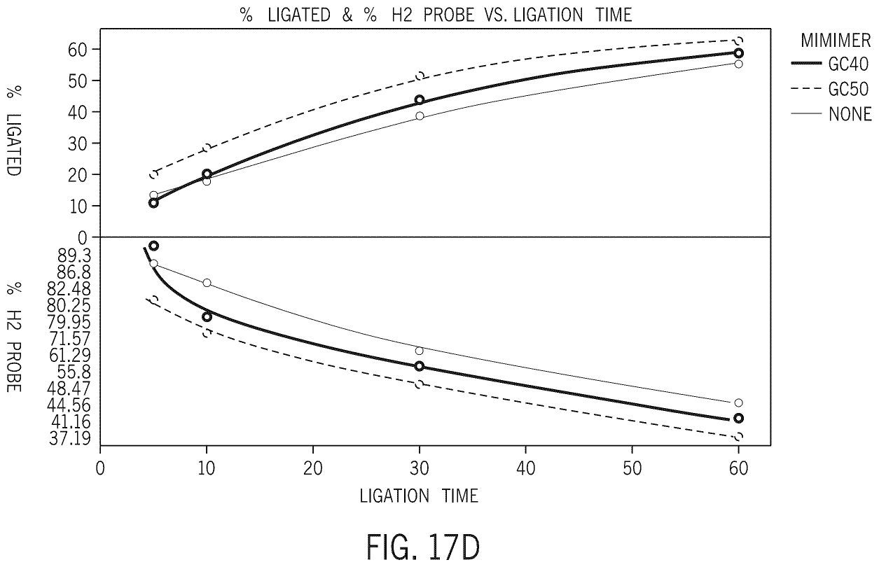

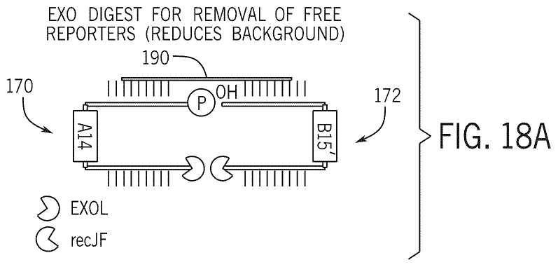

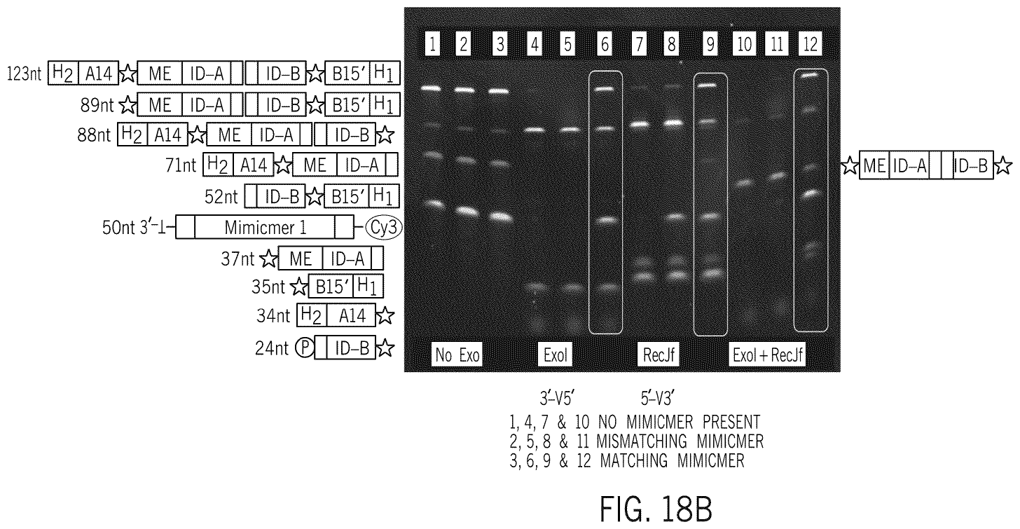

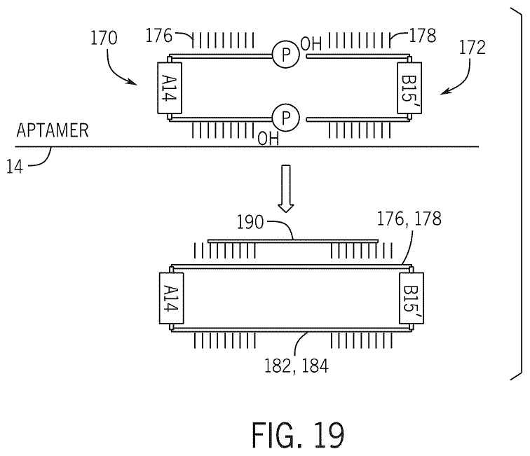

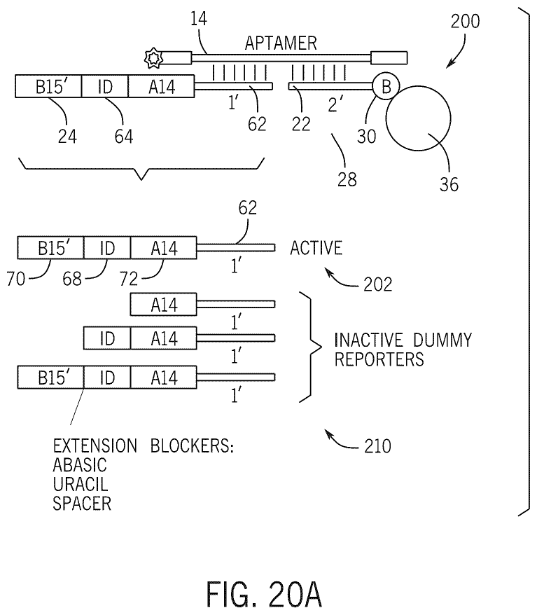

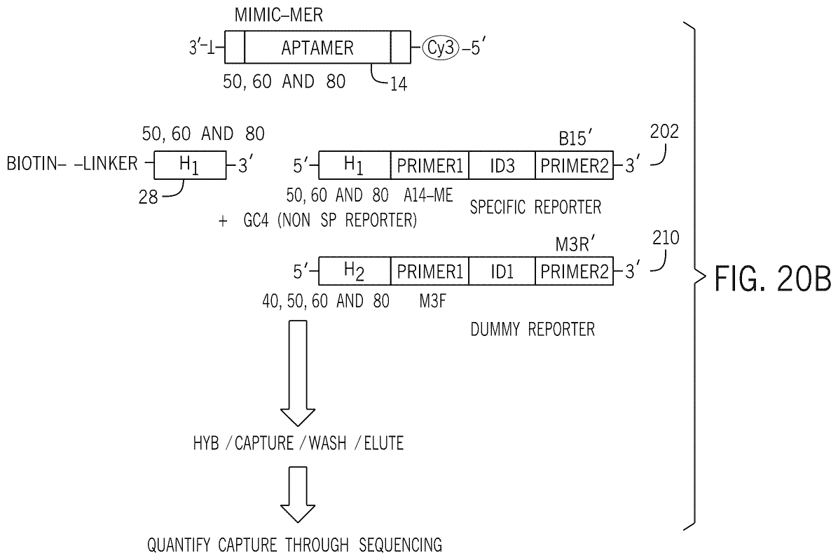

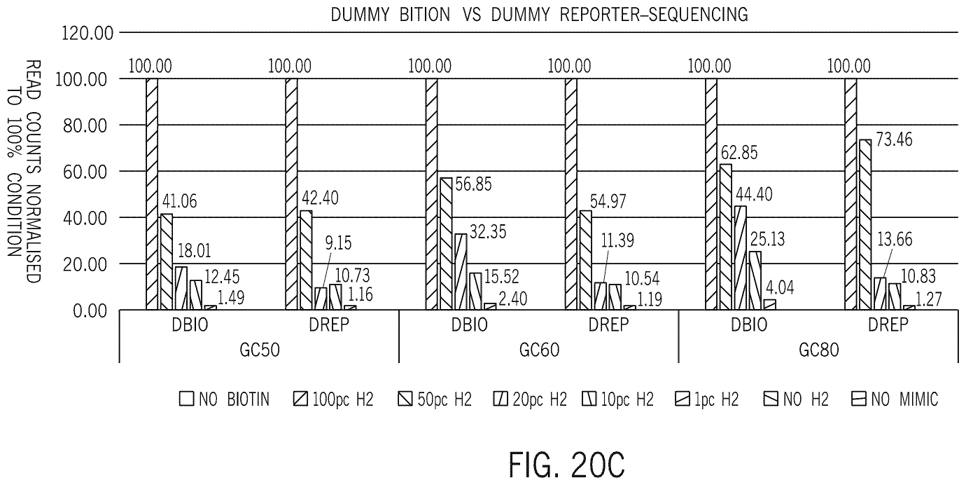

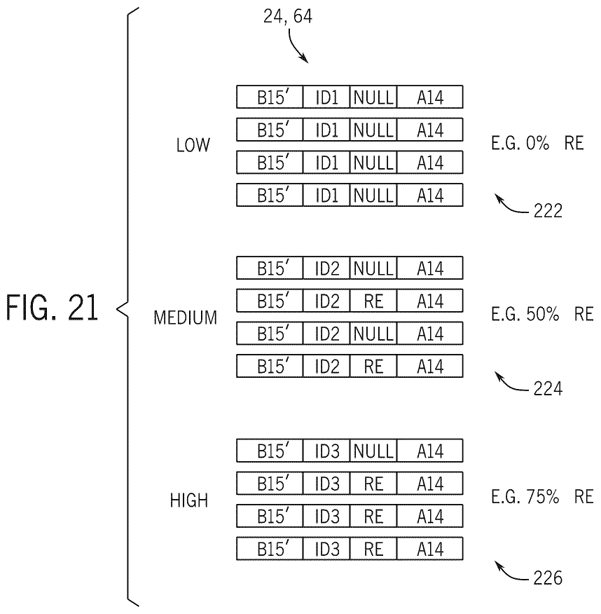

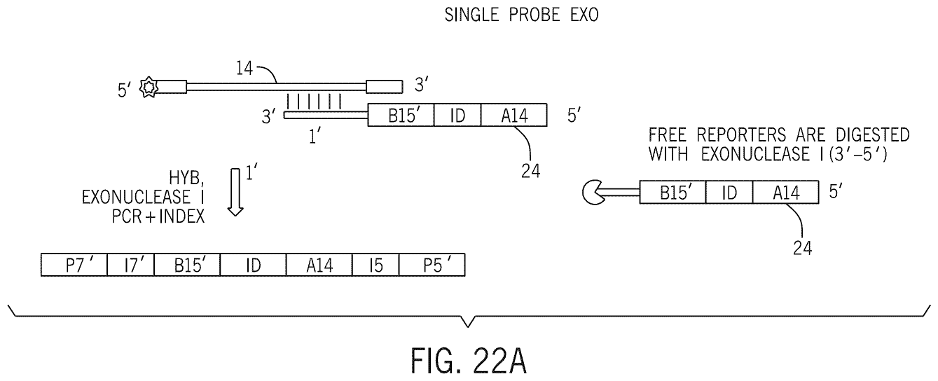

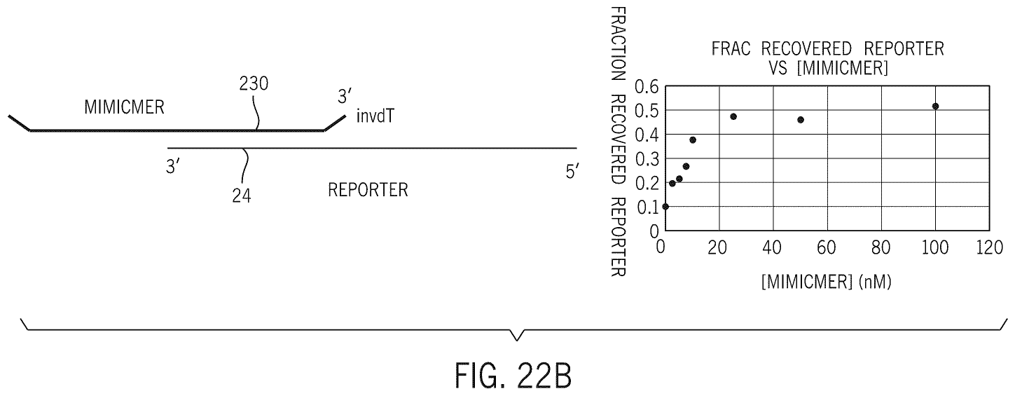

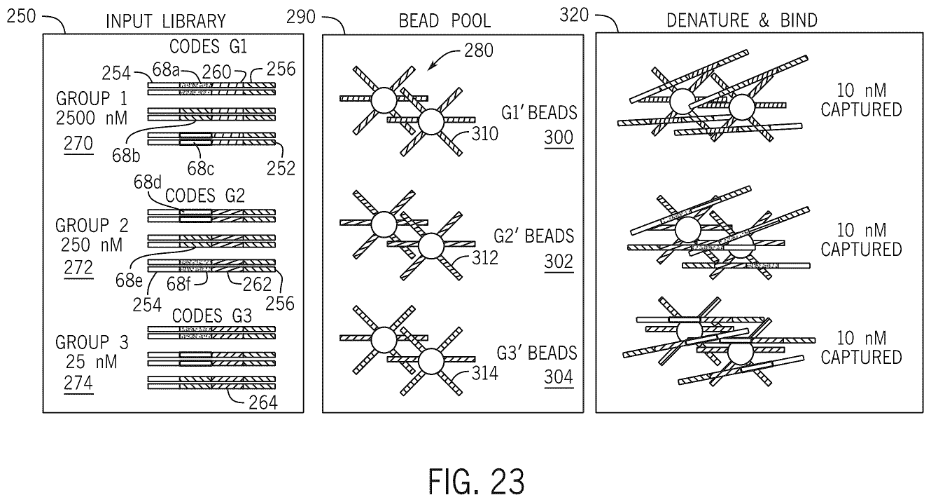

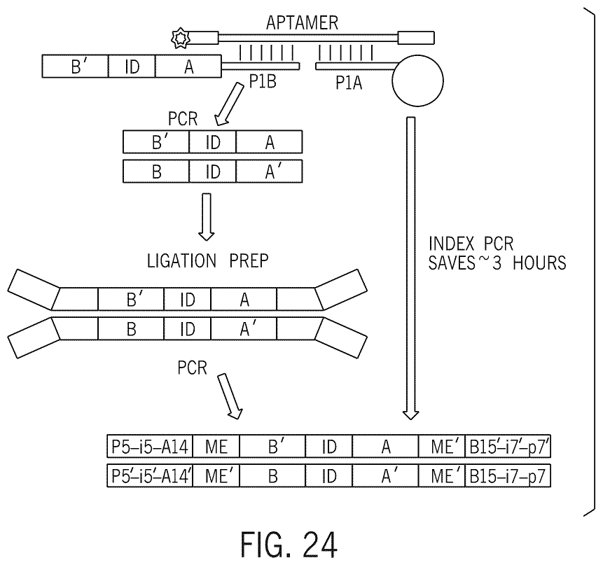

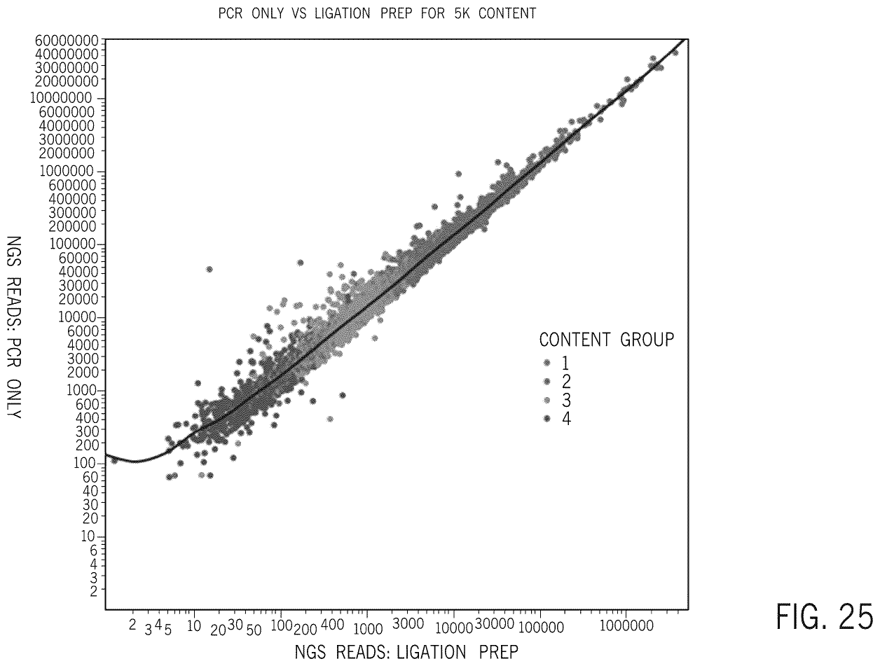

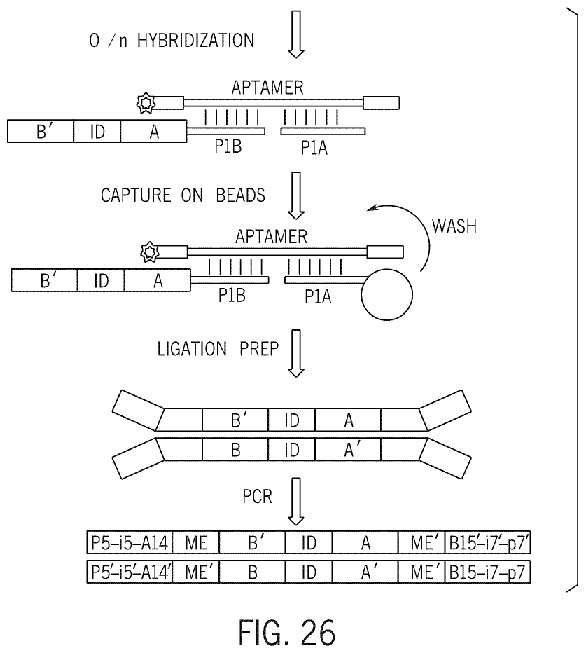

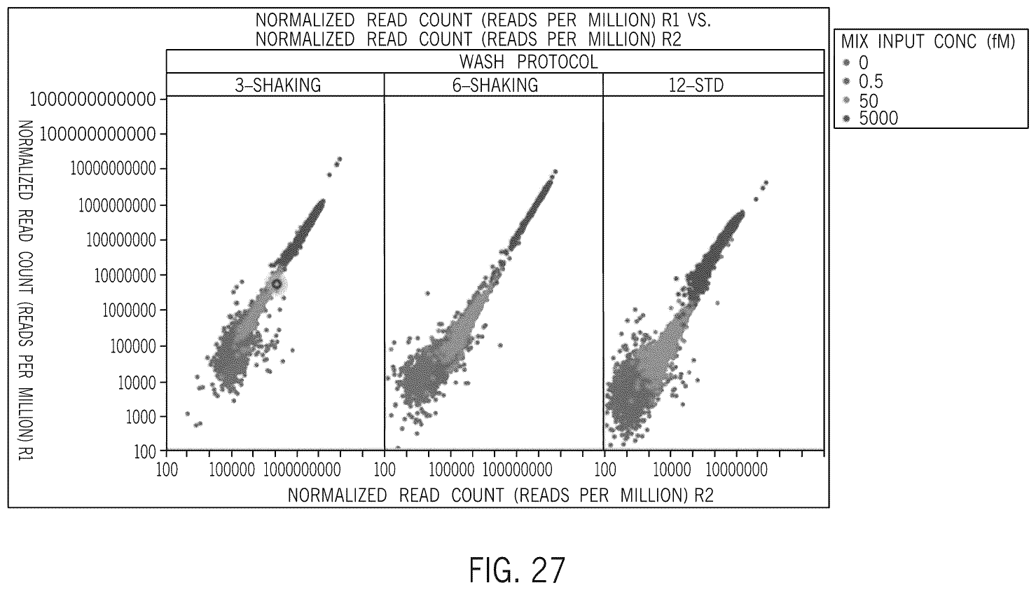

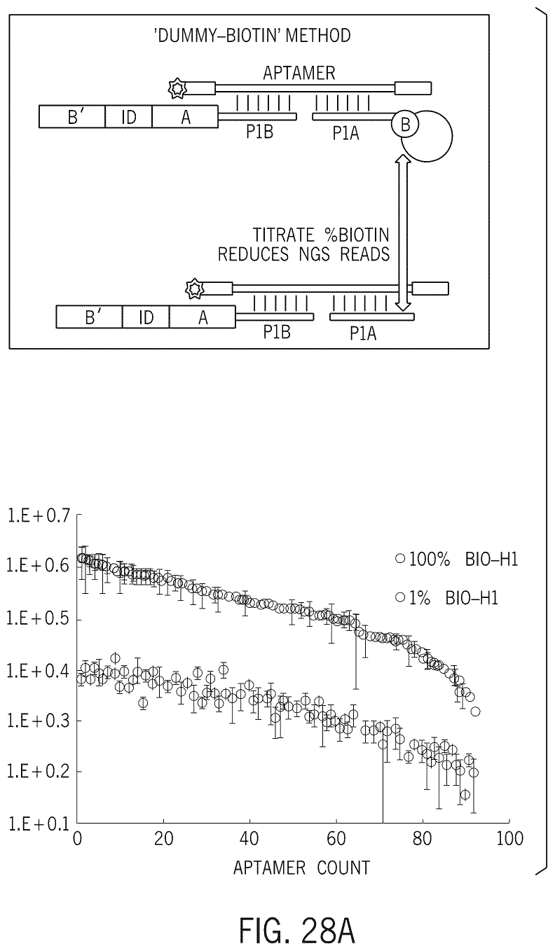

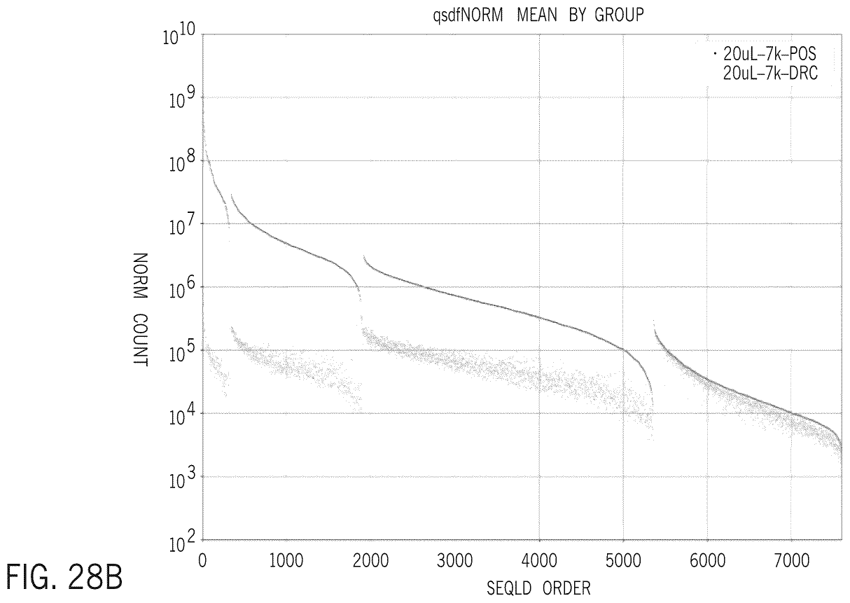

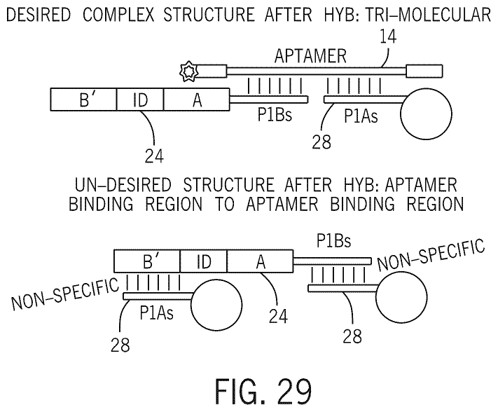

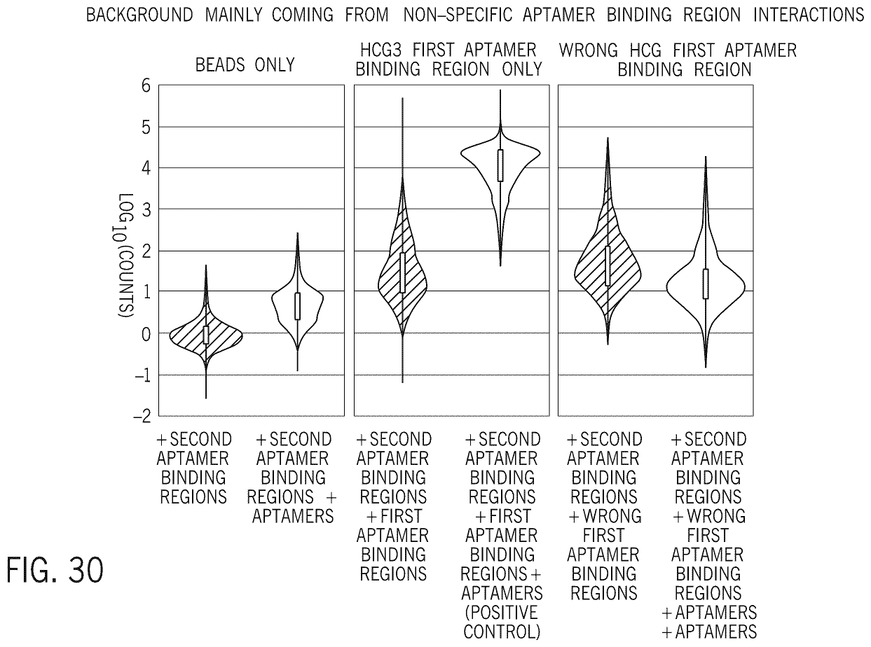

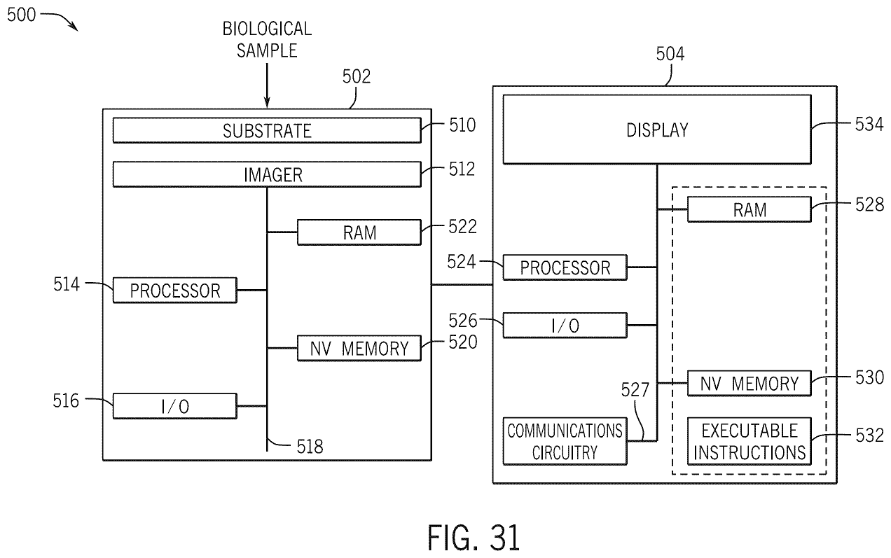

FIG. 8C shows a tapestation of amplification products of Option 2 of FIG. 9A;

FIG. 10 shows example sequencing from step out amplification techniques, according to an embodiment;

FIG. 11 shows example reporter probe ligation amplification techniques, according to an embodiment;

FIG. 12 shows example sequencing from ligation amplification techniques, according to an embodiment;

FIG. 13 shows an example splint ligation technique, according to an embodiment;

FIG. 14A shows an example extension ligation technique, according to an embodiment;

FIG. 14B shows PCR-free library conversion of an extension ligation technique;

FIG. 14C shows conversion efficiency of an extension ligation technique;

FIG. 15 shows an example extension ligation technique, according to an embodiment;

FIG. 16 shows an example split reporter probe technique, according to an embodiment;

FIG. 17A shows an example split reporter probe technique using a splint, according to an embodiment;

17B shows an example split reporter probe technique using a splint, according to an embodiment;

17C shows ligation products of a split reporter probe technique;

17D shows efficiency of generating ligation products over time of a split reporter probe technique;

FIG. 18A shows an example exonuclease digestion for use in conjunction with a split reporter probe technique, according to an embodiment;

FIG. 18B shows ligation product protection in the presence of exonuclease digestion;

FIG. 19 shows an example exonuclease digestion for use in conjunction with a circularized split reporter probe technique, according to an embodiment;

FIG. 20A shows an example dummy reporter technique using a mix of amplifiable and nonamplifiable regions, according to an embodiment;

FIG. 20B shows an example dummy reporter technique using a mix of amplifiable and nonamplifiable regions, according to an embodiment;

FIG. 20C shows sequencing read counts in the presence of dummy reporters;

FIG. 21 shows an example dummy reporter technique using an integral restriction enzyme site, according to an embodiment;

FIG. 22A shows an example exonuclease digestion technique, according to an embodiment;

FIG. 22B shows reporter probe exonuclease digestion;

FIG. 23 shows example bead-based selection techniques, according to an embodiment;

FIG. 24 shows an example streamlined workflow using index amplification, according to an embodiment;

FIG. 25 is a plot comparing sequencing read counts from the streamlined workflow of FIG. 24 versus a ligation preparation workflow;

FIG. 26 shows an example workflow with reduced wash steps, according to an embodiment;

FIG. 27 shows sequencing read counts for different wash conditions;

FIG. 28A shows compression of sequencing read counts using a dummy-biotin for different aptamers;

FIG. 28B shows sequencing of captured reporter probes using different dummy-biotin concentrations;

FIG. 29 shows example undesired nonspecific binding between aptamer binding regions;

FIG. 30 shows contributions of different aptamer binding regions to non-specific binding; and

FIG. 31 is a block diagram of a sequencing device configured to acquire sequencing data, according to an embodiment.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

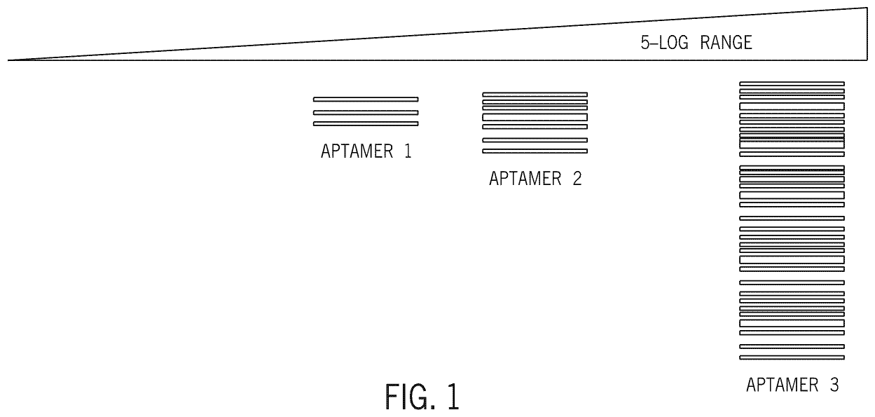

Aptamers are short single stranded nucleic acid molecules (ssDNA or ssRNA) that can bind to their specific target molecules with high affinity. Accordingly, aptamers can be used for multiomic applications, such as proteome characterization of a sample in a high-throughput manner. For assessment of proteins in complex samples in a high-throughput approach, combining aptamers to high-abundancy proteins together with low-abundancy proteins in a single panel is challenging. For example, human serum/plasma contains proteins can differ in concentration by many orders of magnitude, e.g., a 10-log range. Certain aptamer detection platforms can compress the dynamic range of detected proteins. However, even after compression, the dynamic range can nonetheless be relatively large. FIG. 1 illustrates an example 5-log dynamic range within aptamer detection results for a sample and showing three different aptamers with positive binding results along the wide dynamic range. To address complexities of dynamic range, samples may undergo pretreatment or targeted panels are used to measure proteins over a particular range. These approaches add additional complexity and opportunities for loss of low concentration proteins.

Disclosed herein are techniques to compress a dynamic range of aptamers with positive binding results (e.g., that bind to target molecules in a sample) and that may occur before or in conjunction with an aptamer detection step. The techniques preserve the aptamer binding for low-abundancy proteins that are assessed together with high-abundancy proteins. Further, because low-abundancy proteins may correspond to biomarkers that can be used for diagnostic purposes, the disclosed techniques prevent noise or false negative results of an aptamer-based assay caused by high-abundancy proteins obscuring the results. In addition, reducing the dynamic range can also reduce the amount of total sequencing data required to detect aptamers in a detection assay by reducing the amounts of reads wasted on high-abundance aptamer sequences. In certain embodiments, the disclosed techniques may provide streamlined workflows with reduced equipment burden via reduction in a number of steps (e.g., single hybridization reactions or reduced number of wash steps). The disclosed techniques may include sample preparation steps and/or sample preparations that permit improved aptamer abundance measurement.

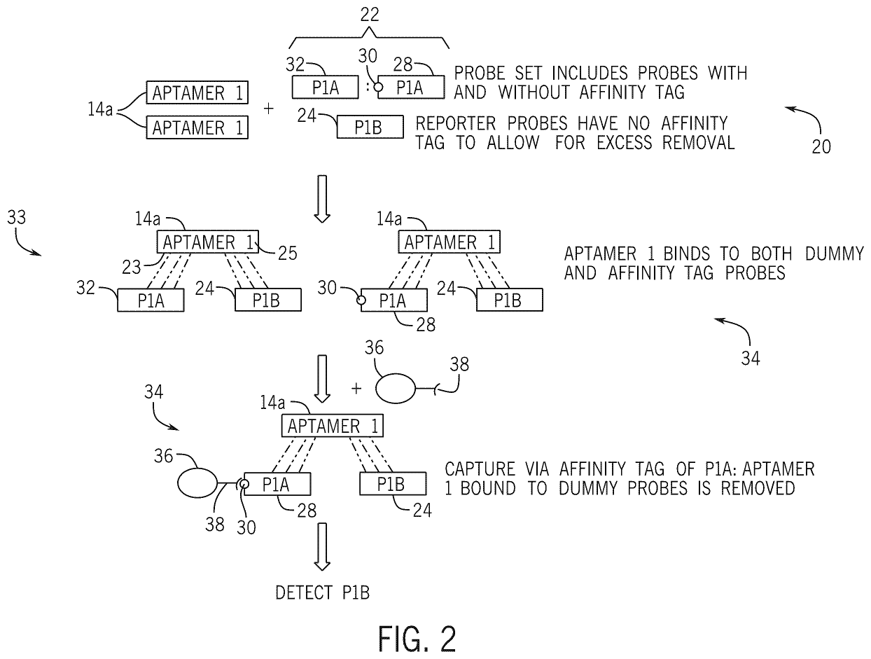

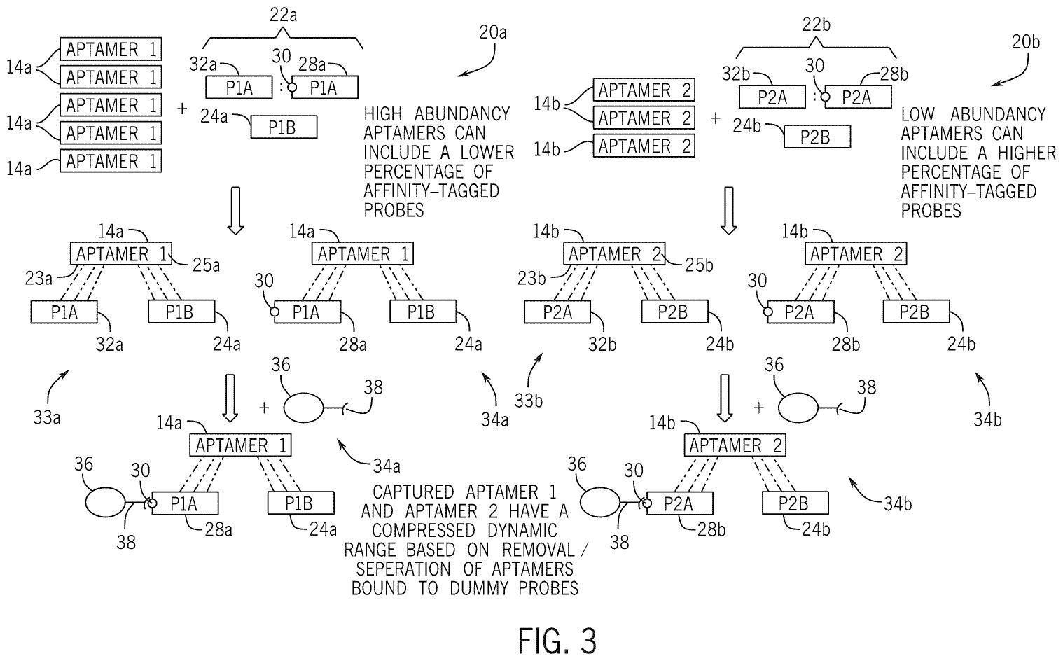

FIG. 2 shows an example workflow for dynamic range compression in which a dynamic range of an individual aptamer 14a can be compressed by removal of some of the aptamer 14a before a detection step. In the illustrated workflow, dynamic range compression for a single aptamer type of an individual aptamer 14a is shown. It should be understood that the illustrated workflow may be extended to all aptamers in a multiplexed aptamer-based assay in parallel. Further, the assay eluate may include multiple aptamers 14a, which is dependent on the concentration of the target molecule of the aptamer 14a in the assessed sample. The aptamer 14a is a single-stranded nucleic acid having a fixed or substantially fixed nucleic acid sequence. Thus, copies or multiples of the individual aptamer 14a may all share a conserved sequence. Different aptamers, referred to generally as aptamers 14 (see FIG. 3), may have different nucleic acid sequences relative to one another, which facilitates different target specificity for respective different aptamers 14.

Using the conserved sequence of the aptamer 14a, a probe set 20 can be designed that includes first probes 22 that hybridize to a first region 23 of the aptamer 14a (e.g., via complementary sequences) and second probes 24 that hybridize to a second region 25 of the aptamer 14a. The first probes 22 are a mixture of at least two different types of probes, both sharing the ability to hybridize to the first region 23. As illustrated, the mixture includes affinity-tagged probes 28 that include an affinity tag 30 and dummy probes 32 lacking the affinity tag 30. In an embodiment, the affinity-tagged probes 28 and the dummy probes 32 are identical other than the presence or absence of the affinity tag 30. The ratio of the affinity-tagged probes 28 to the dummy probes 32 can be tuned based on the abundancy of the target of the aptamer 14a, as generally discussed herein.

The workflow includes a step of contacting the aptamers 14a with the probe set 20, e.g., with the first probes 22 and the second probes 24. Because the affinity-tagged probes 28 to the dummy probes 32 of the first probes 22 both have a same binding ability and specificity for the first region 23 of the aptamer 14a, contact of the first probes with the aptamer 14a results in both the affinity-tagged probes 28 and the dummy probes 32 binding. If the affinity-tagged probes 28 are rare (e.g., less than 10% by way of example) within the mixture of first probes 22, most of the aptamer 14a will be bound to dummy probes 32. Further, all of the second probes 24 can be identical to one another. Thus, two different types of tri-molecular complexes are formed for the aptamer 14a. A first type 33 includes the second probe 24 and the dummy probe 32. A second type 34 includes the second probe 24 and the affinity-tagged probe 28. Again, because the first probes 22 are provided as a mixture, the relative ratio of the first type 33 and second type 34 of tri-molecular complex is dependent on the ratio of the affinity-tagged probes 28 to the dummy probes 32 in the first probes 22. The ratio of affinity-tagged probes 28 to the dummy probes 32 can be selected for each aptamer in the assay based on its relative abundance to the other aptamers to compress the dynamic range for downstream detection, e.g., via NGS.

The workflow also includes a step of separating the first type 33 of tri-molecular complex from the second type 34 of tri-molecular complex via a capture entity. For example, only the second type 34 of tri-molecular complex can be captured using the capture entity, illustrated here as a capture bead 36 coupled to an affinity tag binder 38. However, other arrangements are also contemplated, including column-based, flow-cell based, or substrate-based separation using a capture entity that binds to the affinity tag 30. The unbound first type 33 can be washed or separated, leaving only the second type 34 of tri-molecular complex and its component molecules, the aptamer 14a, the affinity-tagged probe 28, and the second probe 24. In addition, unbound or uncaptured probes of the probe set 20 are also removed. The workflow also includes detection, such as via sequencing, of the second probe 24 or oligonucleotides amplified or otherwise derived from the second probe 24, as a proxy measure of the aptamer 14a as generally discussed herein.

FIG. 3 shows an example workflow for dynamic range compression comparing a high abundancy aptamer 14a to a low abundancy aptamer 14b. For example, high abundancy aptamers 14a may have specific binding affinity for proteins that are known to be abundant, such as albumin, α-2-Macroglobulin, Apolipoprotein A1, Complement C4, IgGs, IgMs, Apolipoprotein A2, α-1-Antitrypsin, Plasminogen, or collagen. Low-abundancy aptamers 14b may have specific binding affinity for biomarkers, transiently-expressed proteins, proteins expressed in only a certain type of cell, etc. It should be understood that these are examples, and that the identity of protein targets is dependent on the composition of aptamers in the aptamer-based assay. Further, it should be understood that, in certain embodiments, a high abundancy aptamer and a low abundancy aptamers may be based on abundancy relative to one another, or other aptamers in an aptamer-based assay, rather than absolute abundance or concentration.

In the illustrated example, the high-abundancy aptamer 14a can be expected to be present at a higher concentration on an aptamer-based assay eluate relative to the low-abundancy aptamer 14b based on, for example, empirical studies or retrospective analysis. Thus, to compress the dynamic range at the downstream detection step, different mixtures of first probes in the probe set 20a, 20b can be used based on the predicted abundancy. For the high-abundancy aptamer 14a, relatively more of the aptamer-bound dummy complexes can be removed via binding to the dummy probe 32a. Thus, the dummy probes 32a can be present in a higher percentage in the first probes 22a. To convert less of the aptamer 14b via dummy binding prior to the detection step, the dummy probes 32b can be present in a relatively lower percentage in the first probes 22b. In one embodiment, the percentage of dummy probes 32b can be 0%. That is, for certain aptamers, the probes 22 can only include tagged probes 28 and include no dummy probes 32. Thus, the ratio of the dummy probes 32 to the affinity-tagged probes 28 can be tuned and can be different for different aptamers 14. In a high-throughput assay, each individual aptamer 14 can be associated with a different ratio of dummy probes 32 to the affinity-tagged probes 28 in an embodiment.

In embodiments, the ratio of the dummy probes 32 to the affinity-tagged probes 28 in the mixture of first probes 22 can be more than more than 100,000:1, more than 10,000:1, more than 1000:1, more than 100:1, more than 20:1, more than 10:1, more than 5:1, more than 2:1, about 1:1, less than 1:2, or less than 1:5. In an embodiment, the mixture of first probes 22 only includes dummy probes 32 or affinity-tagged probes 28, and no other probe types. In embodiments, the dummy probes 32 are at least 25%, at least 50%, at least 75%, or at least 90% of the mixture of first probes 22. In an embodiment, the mixture of first probes 22 only includes dummy probes 32 or affinity-tagged probes 28, and no other probe types. In an embodiment, the first probes 22 only include affinity-tagged probes 28 and do not include any dummy probes 32. For example, for very low abundancy proteins, it may not be desirable to lose any aptamer 14 via removal.

In a high-throughput assay, each individual aptamer 14 can be associated with a different ratio of dummy probes 32 to the affinity-tagged probes 28 in an embodiment such that each individual aptamer 14 has a unique ratio relative to other aptamers 14 used together in a panel or assay. In an embodiment, certain groups of aptamers 14 all associated with an approximate abundancy range can have a same ratio of dummy probes 32 to the affinity-tagged probes 28 relative to one another. In embodiments, for a high-throughput assay, at least 3 different ratios of dummy probes 32 to the affinity-tagged probes 28 are present for a group of at least 1000 different aptamers 14. In embodiments, at least 5, 10, 50, 100, or more different ratios of dummy probes 32 to the affinity-tagged probes 28 are present for aptamers 14 of an assay.

The workflow includes the step of contacting the aptamers 14a, 14b with the probe sets 20a, 20b e.g., with the first probes 22a, 22b and the second probes 24a, 24b. It should be understood that the first probes 22a, 22b have binding ability and specificity for the different first regions 23a, 23b, and, therefore, have different nucleic acid sequences. Similarly, the second probes 24a, 24b have binding ability and specificity for the different second regions 25a, 25b and, therefore, have different nucleic acid sequences. Contact with the probe sets 20a, 20b causes formation of tri-molecular complexes of the first type 33a, 33b and the second type 34a, 34b. Thus, in the illustrated example, because of the different ratios of dummy probes 32 to the affinity-tagged probes 28 in the first probes 22a, 22b relative to one another, different ratios of the first type 33a, 33b of tri-molecular complex and the second type 34a, 34b of tri-molecular complex are formed between the different aptamers 14a, 14b. Because aptamer 14a is more abundant, a greater percentage of the first type 33a can be formed and, subsequently, removed, at the capture step using the affinity tag 30 and the capture entity, e.g., the capture bead 36 and affinity tag binder 38. The affinity tag 30 can be a same tag for all affinity-tagged probes 28, permitting capture of all of the second typed 34 of tri-molecular complexes in a same manner.

It should be understood that, in embodiments, for the high-abundancy aptamer 14a, even if the majority of the complex formation is of the first type 33a such that at least 50%, at least 75%, or at least 90% is removed, the high-abundancy aptamer 14a may nonetheless be present in greater amounts at detection, simply due to the higher overall starting concentration relative to the low-abundancy aptamer 14b. That is, 1% of the high-abundancy aptamer 14a may be greater than 100% of the low abundancy aptamer 14b. However, the disclosed techniques can compress the dynamic range by one log, two logs, or more based on tuning of the ratios or other techniques as discussed herein.

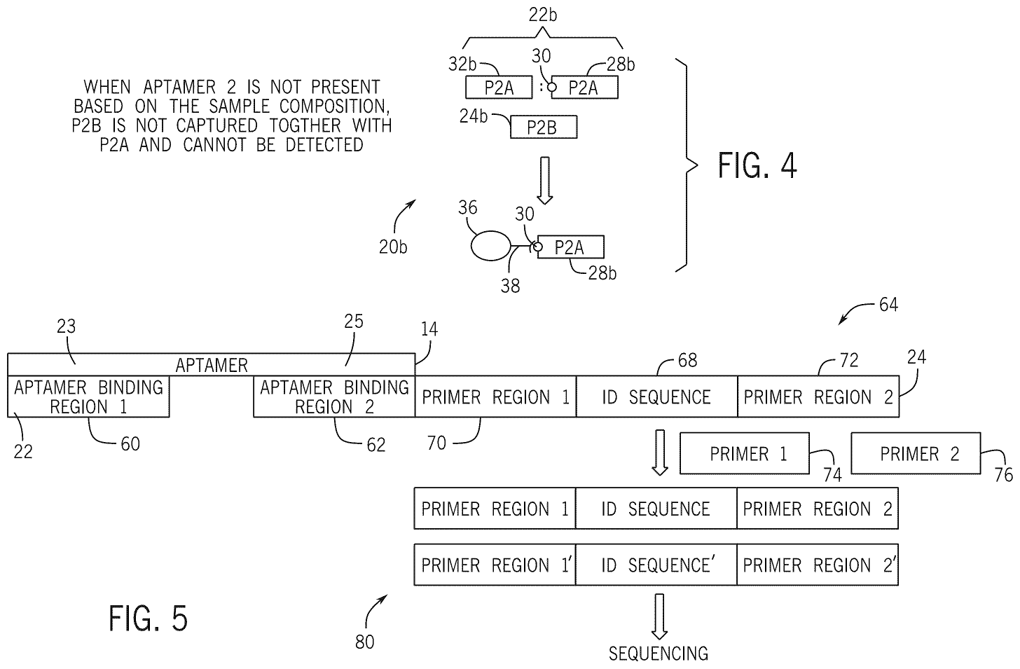

The disclosed techniques include workflow in which tri-molecular complexes are formed, and an affinity-tagged probe 28 used to capture the aptamer 14 is separate from a second probe 24 that is detected. FIG. 4 shows the benefits of separating a reporter probe or detection probe, e.g., the second probe 24b, from the capture probe, e.g., the affinity-tagged probe 28b. In one example, the aptamer 14b is not detected in a particular sample based on the sample composition. Thus, there is no aptamer 14b present in the workflow. In such an example, during capture of other tri-molecular complexes, e.g., from the aptamer 14a, via the affinity-tagged probe 28. The capture bead 36 can pull down the affinity-tagged probe 28b. However, the absence of the aptamer 14b to bridge the gap and bind to the second probe 24b, means that there is no second probe 24b to be detected. If the detectable moiety were on the affinity-tagged probe 28b, the illustrated example would yield a false positive.

FIG. 5 is a schematic illustration of a tri-molecular complex, which may be of the first type 33 or the second type 34, depending on the type of bound first probe 22 (e.g., the affinity-tagged probe 28 or the dummy probe 32) as generally discussed herein. The first probe 22 hybridizes to the first region 23 of the aptamer 14 via a first complementary region 60, e.g., a first aptamer binding region. The second probe 24 hybridizes to the second region 25 of the aptamer 14 via a second complementary region 62, e.g., a second aptamer binding region. The first complementary region 60 and the second complementary region 62 are unique to each individual aptamer 14. It should be understood that the relative arrangement of the first probe 22 and the second probe 24 on the aptamer 14 can be exchanged, such that the first probe 22 may be 5′ of or 3′ of the second probe 24. The first region 23 and the second region 25 can be spaced apart from one another on the aptamer 14, e.g., by at least 1-2 nucleotides. In an embodiment, the first region 23 and the second region 25 are spaced apart from one another by 1-30 nucleotides. Providing spacing may provide benefits such as normalizing melting temperatures between prove sets of different aptamers 14 or reducing nonspecific complementarity.

The first region 23 and the second region 25 can be contiguous or adjacent to one another, e.g., with zero nucleotide separation. A contiguous arrangement of the first probe 22 and the second probe 24 may facilitate workflows in which the first probe 22 and the second probe 24 are ligated to one another, e.g., directly ligated at respective ends, subsequent to aptamer binding. In an embodiment, the first probe 22 and/or the second probe 24 may include matched overhangs or may be blunt end, depending on the desired ligation protocol. Ligation of the first probe 22 to the second probe 24 can provide the advantage of reducing variance of melting temperatures between the sets of different probes used in a workflow and can also avoid the need for Tm enhanced probes. Further, ligation can facilitate higher stringency washes for greater background removal and/or a reduced number of washes for streamlined workflow. In an embodiment, a ligation-based approach may also contribute to dynamic range compression. For example, the first probe 22 and/or the second probe 24 may be provided as a mixture with dummy probes. In an embodiment, the second probe 24 may be provided as a mixture including both a ligatable version that includes a 5′ phosphate for ligation and a nonligatable version, having a same sequence and aptamer binding capability as the ligatable version, but without the available 5′ phosphate. The ratio of the nonligatable version and the ligatable version may be tuned based on aptamer abundance. Highly abundant aptamers may be provided with a probe mixture having less of the ligatable version in the mixture relative to aptamers of lower abundance. After ligation to available ligatable version, the melting temperature and binding of the ligated product would be higher. Thus, higher stringency washes would result in retention of the ligated product and loss of the non-phosphorylated but bound nonligatable version. In one embodiment, the ligated probes can be protected and separated from nonligated reporter probes 24 with a 5′ affinity reagent such as biotin bound to streptavidin on the beads, and free probes can be digested using an exonuclease, as discussed in FIG. 19, while the ligated probes are protected from exonuclease digestion.

The second probe also includes a nonhybridizing region 64 that extends away from the second complementary region 62 and that does not hybridize to the aptamer 14. Thus, the sequence of the nonhybridizing region 64 can be selected to avoid substantial complementarity with a sequence of the aptamer 14. The nonhybridizing region 64 can be used for detection as a proxy for the aptamer 14. Accordingly, the nonhybridizing region 64 can include a bar code or identification sequence 68 that is unique to the individual aptamer 14. Thus, different aptamers 14 are associated with respective different identification sequences 68 that are all different from one another and are uniquely identifying. In an embodiment, uniquely identifying sequences are uniquely identifying while accounting for barcode errors (e.g., a 1-2 nucleotide sequence error) during sequencing. Further, the identification sequence 68 may be designed such that the identification sequence 68 is different from the aptamer sequence.

To facilitate detection, the nonhybridizing region 64 can include a first primer region 70 and a second primer region 72 that flank the identification sequence 68 such that amplification of the nonhybridizing region 64 using primers 74, 76, to generate amplification products 80 as generally discussed herein, will amplify the identification sequence 68 to permit detection of the aptamer 14. In an embodiment, the amplification is part of preparation of a sequencing library for sequencing.

Because the nonhybridizing region 64 is single-stranded, the first primer region 70 can represent a primer binding site that is a reverse complement of a first primer 74, while the second primer region 72 can correspond to the sequence of a second primer 76 that binds to an amplified strand generated from the first primer 74.

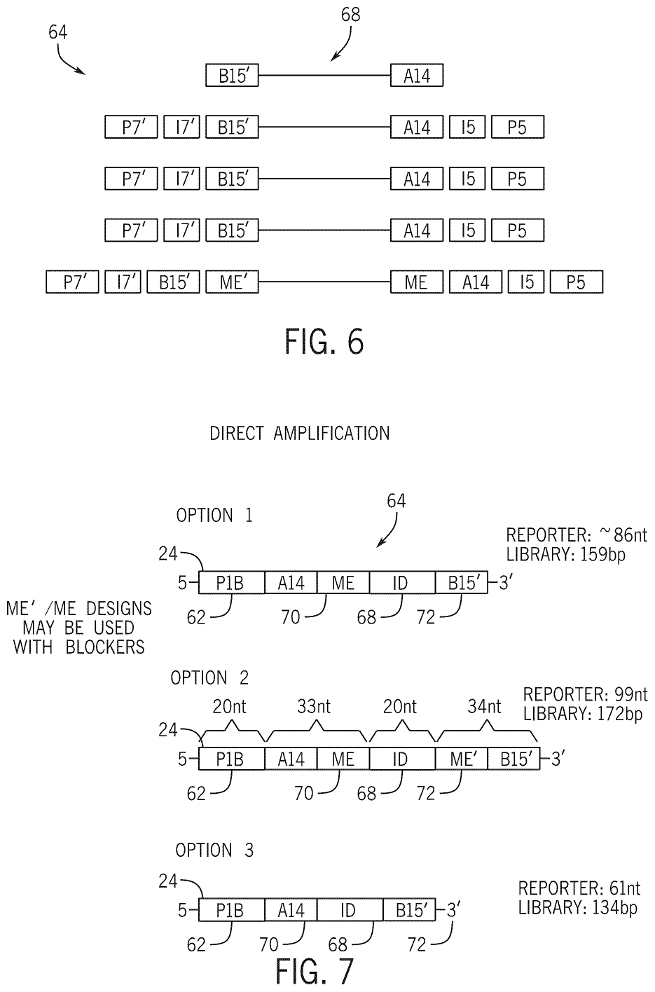

FIGS. 6-15 show different embodiments of amplification techniques, ligation techniques, and/or sequencing techniques and corresponding arrangements of the nonhybridizing region 64 that can be used to conform the generated amplification products 80 into inputs for sequencing library preparation or, in embodiments, into a sequencing library that can be sequenced to generate sequence data of the amplification products. Accordingly, the disclosed embodiments may, in embodiments, provide an advantage of incorporating one or more sequencing library preparation steps into the detection of the aptamer 14. Further, the disclosed embodiments may permit certain steps of sequencing library preparation to be omitted or combined, thus increasing detection efficiency. In embodiments, the disclosed embodiments are also directed to sequencing techniques that permit generation of sequence data from sequence reads of the amplification products 80.

FIG. 6 is a schematic illustration of different arrangements of the nonhybridizing region 64 that include universal or conserved sequences that can be used in conjunction with Illumina® sequencing reactions. It should be understood that these are by way of example, and any of the disclosed arrangements may be used in conjunction with disclosed techniques.

A nonhybridizing region 64 can include a minimum sequence of just the primer regions 70, 72 flanking the identification sequence to introduce an adapter sequence, such as examples of sequences, or their complements, for primer 1 and primer 2 used in Illumina® sequencing preparations, A14, B15, during amplification. In other embodiments, universal capture primer sequences and/or sample index sequences can be incorporated into oligonucleotides generated from the reporter probes 24, such as via amplification and/or ligation and extension. Certain arrangements that include indexes may incorporate a custom or bridged primer during sequencing to accommodate the different indexes. Other embodiments may include custom options for sequencing libraries using single reads from surface P5 for example, or for adding dark sequencing by synthesis cycles where common sequences exist in adapter regions.

The adapter sequences A14-ME, ME, B15-ME, ME′, A14, B15, and ME are provided below:

A14-ME:
(SEQ ID NO: 1)
5′-TCGTCGGCAGCGTCAGATGTGTATAAGAGACAG-3′
B15-ME:
(SEQ ID NO: 2)
5′-GTCTCGTGGGCTCGGAGATGTGTATAAGAGACAG-3′
ME′:
(SEQ ID NO: 3)
5′-phos-CTGTCTCTTATACACATCT-3′
A14:
(SEQ ID NO: 4)
5′-TCGTCGGCAGCGTC-3′
B15:
(SEQ ID NO: 5)
5′-GTCTCGTGGGCTCGG-3′
ME:
(SEQ ID NO.: 6)
AGATGTGTATAAGAGACAG

The primer region or primer binding region can include a region having the sequence of a universal Illumina® capture primer or a region specifically hybridizing with a universal Illumina® capture primer. Universal Illumina® capture primers include, e.g., P5 5′-AATGATACGGCGACCACCGA-3′ ((SEQ ID NO: 7)) or P7 (5′-CAAGCAGAAGACGGCATACGA-3′ (SEQ ID NO: 8)), or fragments thereof. A region specifically hybridizing with a universal Illumina® capture primer can include, e.g., the reverse complement sequence of the Illumina® capture primer P5 (“anti-P5”: 5′-TCGGTGGTCGCCGTATCATT-3′ (SEQ ID NO: 9) or P7 (“anti-P7”: 5′-TCGTATGCCGTCTTCTGCTTG-3′ (SEQ ID NO:10)), or fragments thereof.

A conserved primer region can additionally or alternatively include a region having the sequence of an Illumina® sequencing primer, or fragment thereof, or a region specifically hybridizing with an Illumina® sequencing primer, or fragment thereof. Illumina® sequencing primers include, e.g., SBS3 (5′-ACACTCTTTCCCTACACGACGCTCTTCCGATCT-3′ (SEQ ID NO: 11)) or SBS8 (5′-CGGTCTCGGCATTCCTGCTGAACCGCTCTTCCGATCT-3′ (SEQ ID NO: 12)). A region specifically hybridizing with an Illumina® sequencing primer, or fragment thereof, can include, e.g., the reverse complement sequence of the Illumina® sequencing primer SBS3 (“anti-SBS3”: 5′-AGATCGGAAGAGCGTCGTGTAGGGAAAGAGTGT-3′ (SEQ ID NO: 13)) or SBS8(“anti-SBS8”:

5′-AGATCGGAAGAGCGGTTCAGCAGGAATGCCGAGACCG-3′ (SEQ ID NO: 14)), or fragments thereof. The incorporation of sequencing primer sequences in the reporter probes may be either directly or via subsequent amplification, ligation, or other sequencing library preparation steps.

In an embodiment, the disclosed amplification products 80 may include amplification products that differ from one another based on different identification sequences 68 but that have conserved or universal primer regions 70, 72. In this manner, a single primer set can be used to amplify reporter probes 24 that have variable identification sequences 68. Provided herein are library preparation kits that include primers 74, 76 that are capable of generating the amplification products 80 from the reporter probes 24 to generate sequencing libraries. The sequence of the primers 74, 76 is based on the sequencing of the first primer binding region 70 and the second primer binding region 72. However, it should be understood that these arrangements are by way of example, and the primer regions 70, 72 for primer binding may be selected to be compatible with other library preparations.

In an embodiment, the sequencing may use Illumina® NGS primers. The following primers are shown by way of example.

Read 1
(SEQ ID NO: 15)
5′ TCGTCGGCAGCGTCAGATGTGTATAAGAGACAG 3′
Read 2
(SEQ ID NO: 16)
5′ GTCTCGTGGGCTCGGAGATGTGTATAAGAGACAG
Paired End Read
(SEQ ID NO: 17)
15′ ACACTCTTTCCCTACACGACGCTCTTCCGATCT
Paired End Read 2
(SEQ ID NO: 18)
5′ CGGTCTCGGCATTCCTGCTGAACCGCTC
TTCCGATCT
Index 1 Read
(SEQ ID NO: 19)
5′ CAAGCAGAAGACGGCATACGAGAT[i7]
GTCTCGTGGGCTCGG
Index 2 Read
(SEQ ID NO: 20)
5′ AATGATACGGCGACCACCGAGATCTAC
AC[i5]TCGTCGGCAGCGTC

It should be understood that the index read primers may be designed to include the particular index sequence associated with a particular sample in an aptamer-based assay. Thus, the index primers may have a nucleotide region, shown as i5 or i7, that varies in sequence between different samples of a multiplexed sample. Other samples in the run can be prepared with primers that include their respective indexes. Accordingly, certain sequence reads may be obtained with universal primers while other sequence reads are obtained with primers or a mix of primers that are specific to indexes of one or more samples in a multiplexed reaction.

In an embodiment, unique molecular identifiers (UMIs) may be incorporated onto the reporter probes 24, e.g., via ligation. UMIs are short sequences used to uniquely tag each molecule in a sample library to provide error correction and reduce sequencing bias.

FIG. 7 shows example arrangements of reporter probes 24 for direct amplification via the primers 74, 76 (see FIG. 5). In Option 1, the reporter probe 24 includes both the second complementary region 62 that binds to the aptamer, and the nonhybridizing region 64. The nonhybridizing region 64 includes the first primer region 70 with ME and A14 sequence and the second primer region 72 with the complement of B15 sequence to generate amplification products that can be used with Illumina® sequencing primers. Therefore, their inclusion permits standard Illumina® sequencing or NGS techniques to be performed. The first primer region 70 and the second primer region 72 flank the identification sequence 68. In Option 2, the second primer region 72 includes the ME′ sequence. In Option 3, the ME and ME′ sequences are excluded. Options 1, 2, and 3 provide different length options for the reporter probes 24 as well as different length options for the amplification products. In certain embodiments, smaller reporter probes 24 may be less costly to manufacture and purify, as in Option 3. However, the exclusion of the ME and ME′ sequence may involve nonstandard sequencing techniques, as discussed with respect to FIG. 8.

Example sequencing techniques based on amplification products 80 of the reporter probes of FIG. 7 are shown in FIG. 8A. The reporter probes 24 are directly amplified with appropriate primers 75, 76 such that the amplification products 80 include the desired adapter sequences, including P5, P7, i5, and i7, that are compatible with Illumina® NGS techniques. Thus, the prepared sequencing library, e.g., the amplification products 80 in the illustrated example, are longer than the reporter probes 24. Further, the amplification products 80 may eliminate or exclude the second complementary region 62. In certain embodiments, the amplification products 80 as provided herein may be single or dual-indexed. Each individual sample subjected to an aptamer-based assay may be uniquely associated with a particular index or indexes that are not used for other samples in a multiplexed reaction. A sequence reaction based on the amplification products 80 from Option 1 can use standard sequencing primers and may generate sequence data using a read 1 primer to generate a sequence read that includes the identification sequence 68 as well as index information. In certain cases, additional index information may be obtained from a complementary strand index read using an i5 or other index primer. Similarly, the sequence data from Option 2 amplification products 80 can generate an identification sequence read as well as a first index read. In Option 2, a second index read may also be performed. Index reads may be generally shorter cycle reads. In the illustrated embodiments (e.g., FIG. 8, FIG. 10, FIG. 12), the i5 and R1 primers are A14-ME and A14′-ME′ respectively. The i7 read primer is ME-B15.

Lack of ME sequences, as in Option 3, may involve nonstandard sequencing. In the illustrated example, the index information as well as the identification sequence can be obtained from a single sequence read using a p5 primer by way of example. However, certain cycles are run as dark cycles, e.g., chemistry only in which no images are taken and/or analyzed. Accordingly, certain sequencing embodiments may be used in conjunction with specific operating instructions for a sequencing device, as discussed with respect to FIG. 31.

FIG. 8B shows sequencing data from sequencing libraries created by PCR amplifying using Option 1 of FIG. 8A an oligonucleotide mixture containing different reporter probes. 384 separate dual i5 and i7 index PCR primers were used for the amplification. The libraries were purified by SPRI, quantitated and sequenced as either 24 plex (24 different i5 and i7) or 192 plex (192 different i5 and i7) on NovaSeqX at either 90 or 100 pM loading concentration. An example % base plot is shown, showing the expected reads for the Read 1 (SOMA-iD), Read 2 (i7 read) and Read 3 (i5 read). Sequencing quality metrics including % PF and Q30 data are shown in the table of FIG. 8C.

FIG. 9A shows an example of a step-out PCR in which multiple amplifications and primers can be used to add adapters or other sequences. Option 1 shows a first round amplification to add a 3′ adapter, while the 5′ adapter is completed via a second PCR round. Option 2 shows a reverse orientation. Option 3 shows two step PCR for both the 5′ and 3′ adapter sequences. The reporter probe 24 includes certain sequences within the primer regions 70, 72 that are encompassed with the adapter sequences. The step-out PCR can be conducted in index PCR or in separate reactions. FIG. 9B shows data from amplification of a reporter probe according to Option 1 of FIG. 9A. An identification-sequence containing long oligo with a non-amplified aptamer binding region (P1B) was used as a template in a PCR reaction with 3 primers. The B15 containing primer serves as the ‘fwd’ primer and the two A14 containing primers serve as ‘rev’ primers. Amplification products were loaded on a tapestation for analysis. Decreasing the concentration of the A14-ME primer results in more amplification of the full length product (0.1×A14ME 153 bp-blue) as shown in the tapestation trace. FIG. 9C shows tapestation results for amplification of reporter probes according to Option 2 of FIG. 9B. The P1B-containing long oligo was used as a template in a PCR reaction with 4 primers. The two M13F containing primers serve as the ‘fwd’ primers and the two M13R containing primers serve as ‘rev’ primers. Amplification products were loaded on a tapestation for analysis. The expected product is observed at 130 bp, and decreases with less DNA input. A primer dimer is created at 98 bp in the absence of a DNA template.

FIG. 10 shows sequencing workflows to sequence a library prepared from the amplification products 80 from step-out PCR. A sequence reaction based on the amplification products 80 from Option 1 and Option 2 can use standard sequencing primers and may generate sequence data using a read 1 primer to generate a sequence read that includes the identification sequence 68. Additional index information may be obtained from a complementary strand index read using an i5 or other index primer. A second index read may also be performed to obtain second index information. In Option 3, the indexes can be obtained from first and second index reads, and a custom primer is used to generate a sequence read including the identification sequence 68. The custom primer sequence read can include dark cycles to skip the nonstandard region of the amplification product 80.

FIG. 11 shows a ligation to PCR example in which a double-stranded terminal adapter is ligated to a complementary template on a 3′ end of the probe 24. In the disclosed example, the length of the reporter probe 24 may be tuned based on the desired downstream detection modality as well as reporter synthesis efficiency. For example, shorter reporter probes 24 may be generally less expensive and more pure. However, shorter reporter probes 24 may also include fewer integral adapters for sequencing requiring non-standard sequencing approaches (e.g., single reads with dark cycles). Option 1 shows a relatively shorter reporter probe 24 that includes a first primer region 70 but that does not include a second primer region 72. Instead, the reporter probe 24 has a short 3 nucleotide tail 84 to which a partially double-stranded adapter 86 can be ligated. Option 2 shows a similar arrangement, but with a longer first primer region 70. The resultant amplification products can then incorporate additional sequences (e.g., indexes, p5, p7) via direct or step out amplification techniques as discussed in FIG. 7 and FIG. 9. However, as shown in FIG. 12, sequencing from the relatively shorter amplification product 80 of Option 1 may involve a custom sequencing primer or standard primers (i5, i7) but with incorporation of 3 dark cycles to accommodate the tail 84. Option 2 shows an alternative arrangement in which standard sequencing primers can be used to generate sequence data using a read 1 primer to generate a sequence read that includes the identification sequence 68. Additional index information may be obtained from one or both of i5 or i7 primers, or other combinations of index primers.

Adapters for sequencing or other assays may be added in subsequent ligation and/or PCR steps. Relatively longer reporters may include integral adapters, but may be more expensive, less pure, and/or, if too long, less feasible to synthesize due to lower yields. Accordingly, in certain embodiments, adapter incorporation via direct or indirect ligation steps may be used to modify a relatively shorter reporter probe 24 that participates in aptamer binding but that does not include the adapter sequences (e.g., index sequences, primer binding sequences, functional sequences). The disclosed adapter ligation techniques may be used in conjunction with the dynamic range compression workflows as provided herein, e.g., using dummy probes or reporters. Further, in certain embodiments, the disclosed adapter ligation techniques as discussed herein may be PCR-free workflows that avoid thermocycling. In an embodiment, a PCR-free workflow provides an advantage of reduced potential amplicon contamination and removing the requirement for separate areas for pre and post PCR working.

FIG. 13 shows an example PCR-free workflow that uses splint ligation technique to add one or more adapters via ligation and extension. While the captured reporter probe 24 has a free 3′ end, the 5′ end includes the binding region 62. In other examples, this region 62 is not retained in amplification products using primers that do not cover the region 62. However, in the illustrated example, the reporter probes 24 has an integral cleavage site 90, e.g., a Uracil cleavage site. Here, the reporter probe 24 is captured as part of a tri-molecular complex. The tri-molecular complex may be generated as generally discussed herein, and the noncaptured reporter probes 24 may be associated with a different type of tri-molecular complex that was not captured based on an absence of an affinity tag to facilitate binding to the capture bead 36.

Once captured, uncaptured components are removed, and the nonhybridizing region 64 can be cleaved to expose a 5′ end. The cleavage may be mediated by cleavage of a U base by uracil-DNA glycosylase. After cleaving, the 5′ adapter 94 ligation can be facilitated by a by a 5′ splint 97 that, when hybridized, forms a partially double-stranded ligation region, and the 3′ adapter 96 ligation can be facilitated by a 3′ splint 98 that forms a partially double-stranded ligation region at the 3′ end. The illustrated dotted arrow is a polymerase extension from B15′ that copies the index using a template i7, to add the index complement to the reporter via extension. The extension could include extension to copy completely the p7′ without ligation entirely by extending from the B15′ end, or may add the p7′ to allow for extension-ligation. The polymerase may be a non-strand displacing and without 5-3′ exonuclease activity. In an embodiment, an Illumina extension ligation mix is used. After ligation, and denaturation of the splints 97, 98, the remaining oligonucleotide can be amplified for detection as generally discussed herein.

FIG. 14A shows an example ligation extension workflow to add one or more adapters via ligation and extension. The workflow includes formation of a trimolecular complex as generally discussed herein that includes binding of both the capture probe 28 via the first complementary region 60 and the reporter probe 24 via the second complementary region 62 to corresponding regions of the aptamer 14. The reporter probe 24 includes the nonhybridizing region 64 that does not hybridize to the aptamer 14 having the identification sequence 68 that is uniquely identifying for the aptamer 14. The workflow also includes a step of separating the tri-molecular complex from dummy-containing tri-molecular complexes (see FIG. 1) and/or free aptamers 14 or free reporter probes 24 using a capture entity, such as an affinity tag binder that binds to the affinity tag 30 present on the capture probe 28.

Once captured, the reporter probe 24 and aptamers 14 can be eluted from the capture entity and the capture probe 28. In this workflow, the reporter probe 24 carries a first region 100 that corresponds to a portion of a 5′ adapter sequence and a second region 102 that correspond to a portion of a 3′ adapter sequence. The full 5′ and 3′ adapter sequences may represent respective end adapter sequences that, when present, permit oligonucleotides to be used as part of a sequencing library for NGS sequencing that, in embodiments, may be used to sequencing the identification sequence 68 as part of aptamer detection. In the illustrated workflow, rather having the reporter probe 24 carry the full 5′ and 3′ adapter sequences, the reporter probe carries only part of these sequences and is relatively shorter. For example, the total reporter probe length may be about 70 nucleotides in one example. In embodiments, the reporter probe 24 can be between 50-80 nucleotides. The full 5′ and 3′ sequences are incorporated onto the ends via extension ligation as illustrated.

As illustrated, oligonucleotide 110, carrying a first region complement 111, and oligonucleotide 120, carrying a second region complement 122, hybridizes to the reporter probe 24. The oligonucleotide 110 includes an adapter region 124 that does not hybridize to the reporter probe 24, e.g., is not complementary to the complementary region 62. The oligonucleotide 112 includes an adapter region 130 that does not hybridize to the reporter probe 24 and an affinity tag 30. This hybridization may occur after elution of aptamers from aptamer beads. The oligonucleotide 112 can be extended in a 3′ direction using the identification sequence 68 as a template and ligated to the oligonucleotide 110. In addition, the reporter probe 24 can be extended in a 3′ direction using the adapter region 130 as a template. Thus, the extended reporter probe 24 and the extension ligated oligonucleotides 110, 112 form a partially double-stranded structure that does not hybridize to the complementary region 62. In this manner, the complementary region 62 can be eliminated from the downstream products without a cleavage step, in contrast to the workflow in FIG. 10.

The retained extension ligated oligonucleotide 132 can undergo an additional extension after a wash step (e.g., a hot wash, NaOH, or other denaturant) using a hybridized oligonucleotide 136 as a template. The oligonucleotide 136 hybridizes via a complement region 140 to the adapter region 124. The oligonucleotide 136 also includes a 5′ adapter region 142. In embodiments, the extended hybridized oligonucleotide 136 can be extended and ligated to a hybridized p7′ oligo (not shown) that can be hybridized to the retained extension ligated oligonucleotide 132. The workflow can include 3′ extension of the retained extension ligated oligonucleotides 132 using the 5′ adapter region 142 as a template and 3′ extension of the hybridized oligonucleotide 136 using the extension ligated oligonucleotide 132 as a template.

The oligonucleotides 110, 112 used in the extensions or extension ligation can be universal oligonucleotides that hybridize to any captured (e.g., aptamer-bound) reporter probe 24 via universal regions carried on the reporter probe 24. The oligonucleotide 136 hybridizes to the universal adapter region 124. Thus, the extension ligation oligonucleotide reagents can be used across the panel for aptamer detection.

An optional second capture step can separate the extended oligonucleotide 136 from the extended oligonucleotide 132. Both oligonucleotides 132,136 include full 5′ and 3′ adapters for NGS sequencing or their complements. While the starting reporter probe 24 is shorter (e.g., about 70 nucleotides in one example), the generated product of the extension ligation workflow is longer. In an embodiment, the oligonucleotides 132,136 may be at least 25%, at least 50%, or at least 100% longer than the starting reporter probe 24. The illustrated workflow can be performed with or without subsequent amplification steps in embodiments.

FIG. 14B and FIG. 14C show PCR-free NGS conversion results. A model system was designed to test a PCR-free NGS conversion assay for aptamers. Following hybridization with aptamers, the reporter molecules including identification sequences require addition of indexes and P5/P7 sequences for clustering and sequencing without using PCR. In this model system, the reporter molecules were annealed with three additional oligos, e.g., as in FIG. 14A. The B15 containing oligo allows for extension from the B15′ on the reporter oligo to append i7′ and P7′ to the 3′ end. To append adaptors to the 5′ end of the reporter molecule, a ‘splint’ oligo annealed to the ME portion of the reporter and a second indexing oligo containing A14, i5 and P5. Upon incubating the annealed oligos with ELM (extension ligation mix, Ligase+polymerase, Illumina) the reporter molecule obtains both 5′ and 3′ adaptors containing indexes and P5 and P7′ adaptors respectively. In addition to converting the reporter strand, the method can also create a complete second strand which doubles the yield of the reaction through extension-ligation of the A14′ ‘splint’ oligo and extension of the B15 adaptor across the identification sequence. The addition of the adaptors is quantitated by qPCR as shown in FIG. 14B with a range of fmol inputs of reporter oligo. The output fmol can be used to calculate the conversion efficiency (% LCE) as shown in FIG. 14C and is measured at ˜30% for a wide range of concentrations. In addition to using ELM to perform the conversion, an additional method adds BST polymerase extension as a second step after ELM. This can potentially increase yield by compensating for any failed extension ligation and boost the yield.

FIG. 15 shows another example of a cleavage-free extension ligation technique. As in FIG. 14, the workflow includes formation of a trimolecular complex with binding of both the capture probe 28 via the first complementary region 60 and the reporter probe 24 via the second complementary region 62 to corresponding regions of the aptamer 14. The reporter probe 24 includes the nonhybridizing region 64 that does not hybridize to the aptamer 14 having the identification sequence 68 that is uniquely identifying for the aptamer 14. The workflow also includes a step of separating the tri-molecular complex from dummy-containing tri-molecular complexes (see FIG. 1) and/or free aptamers 14 or free reporter probes 24 using a capture entity, such as an affinity tag binder that binds to the affinity tag 30 present on the capture probe 28.

Once captured, the reporter probe 24 and aptamers 14 can be eluted from the capture entity and the capture probe 28. Multiple oligonucleotide hybridizations form a complex to permit extension ligation of full adapter sequences. The reporter probe 24 includes the first region 100 that corresponds to a portion of a 5′ adapter sequence and the second region 102 that correspond to a portion of a 3′ adapter sequence. As illustrated, oligonucleotide 150, carrying a first region complement 112, and oligonucleotide 152, carrying a second region complement 153, both hybridize to the reporter probe 24. In addition, the oligonucleotide 150 includes an adapter region 155 that does not hybridize to the reporter probe 24, e.g., is not complementary to the complementary region 62, and that carries an internal affinity tag 30. The oligonucleotide 152 includes an adapter region 156 that does not hybridize to the reporter probe 24 and that serves as an extension template. An oligonucleotide 158 hybridizes to the adapter region 155, and an oligonucleotide 160 hybridizes to the oligonucleotide 150 via a region 162. The oligonucleotide 158 acts as a split for ligation of the oligonucleotide 150 and the oligonucleotide 160. In the complex, oligonucleotides 152, 150, 160 can be ligated via extension to form oligonucleotide 166.

The oligonucleotide 166 can be captured via the affinity tag 30, and used as a template for extension of the hybridized oligonucleotide 158. The multiple extensions, e.g., using T4 polynucleotide kinase, permit addition of full 5′ and 3′ adapters. As discussed with respect to FIG. 11, the extension ligation permits use of a shorter reporter probe 24 to generate a longer product, e.g., at least 25%, at least 50%, or at least 100% longer than the starting reporter probe 24. In addition, the oligonucleotides used in the extensions can be universal oligonucleotides that hybridize to any captured (e.g., aptamer-bound) reporter probe 24 via universal regions carried on the reporter probe 24 and can be used across the panel for aptamer detection for different aptamers and their associated different identification sequences 68. The illustrated workflow can be performed with or without subsequent amplification steps in embodiments and with or without additional capture steps. In an embodiment, the extension may be performed from A14 without an initial phosphate blocking.

FIG. 16 shows an example of a workflow using split reporter probes that, together with the aptamer 14, form a trimolecular complex. In contrast to workflows in which the entire identification sequence 68 is provided on a single probe 24, the illustrated example includes a first reporter probe 170 and a second reporter probe 172, and the identification sequence 68 is split between these probes. Using shorter reporter probes is more economical, and the subsequence ligation generates a longer product with library cleanup benefits. Having a split identification sequence distributed between two probes permits assessment of successful hybridization of both probes. This is a benefit because, in other techniques, the second probe is not part of the readout, mishybridization would not be apparent in a results readout.

The first reporter probe 170 carries a first identification sequence 176, and the second reporter probe 172 carries a second identification sequence 178. Similarly, the aptamer binding regions are also split between the probes. The first reporter probe 170 carries a first aptamer binding region 182 and a first primer site 183 positioned between the first aptamer binding region 182 and the first identification sequence 176. The second reporter probe 172 carries a second aptamer binding region 184 and a second primer site 185 positioned between the second aptamer binding region 185 and the second identification sequence 178. The primer sites are shown as truncated or partial adapter sequences (A14′ and B15). It should be understood that additional adapter sequences may also be included in the split probes or may be introduced by amplification and/or ligation as generally discussed herein.

Binding of the first reporter probe 170 and the second reporter probe 172 to the aptamer 14 creates a trimolecular complex, and one of the first reporter probe 170 or the second reporter probe 172 can carry an affinity tag 30, shown as being on the first reporter probe 170 by way of example. The identification sequences and primer sites are carried on nonhybridizing portions of the reporter probes 170, 172. Dynamic range compression can be achieved for split probes by using a mixture that includes a dummy probe (e.g., a dummy first probe 170 or dummy second probe 172) without the affinity tag 30 for certain aptamers 14. As discussed herein, the selected ratio of the dummy to the affinity tag-carrying probe can be tuned based on aptamer abundancy.

The identification sequence 68 can be assembled by ligating the ends of the first reporter probe 170 and the second reporter probe 172, e.g., using a single-stranded ligate, e.g., CircLigase. A 5′ phosphate and adjacent 3′ OH of the probes 170, 172 are ligated together such that the first identification sequence 176 and the second identification sequence 178 are contiguous. The ligated strand can be separated using the affinity tag 30. Any dummy reporter probes 170 and unligated second reporter probes 172 will not be retained. While unligated reporter probes 170 will also be captured, an amplification step using the first primer site 183 and the second primer site 185 ensures that only ligated pairs will generate amplification products. To eliminate false positives from nonspecific or undesired binding, the technique can require a matched pair for the identification sequences 176, 178. That is, the identification sequence 176 and identification sequence 178 can both be identifying for the aptamer 14, and the technique can require a positive sequence match, as assessed using acquired sequencing data from a sequencing device, for both identification sequences 176, 178 before verifying detection of the aptamer 14.

FIG. 17A is an embodiment of the technique of FIG. 16 in which a single-stranded splint oligonucleotide 190 is provided to improve ligation efficiency of ligation of the reporter probes 170, 172. The splint oligonucleotide 190 hybridizes to at least a portion of the first identification sequence 176 and the second identification sequence 178 to create a double-stranded region. When also bound to the aptamer 14, the reporter probes 170, 172 are also partially double-stranded along the aptamer binding regions 182, 184. In an embodiment, the splint oligonucleotide 190 may be between 15-30 nucleotides in length. As shown in FIG. 17B, the reporter probes 170, 172 may include respective terminal conserved or universal sequences 192, 194 that are the same even for reporter probes having different identification sequences 176, 178 such that a common splint oligonucleotide sequence can be used to enhance ligation for a reaction mixture including a panel of different reporter probes 170, 172 forming identification sequences for the full panel of assayed aptamers 14. That is, the reporter probe 170 may include a first terminal sequence 192, and the reporter probe 172 may include a second terminal sequence 194 that is different from the first terminal sequence. However, each different reporter probe 170 may have a different identification sequence 176 relative to one another but share a same terminal sequence 192. Similarly, each different reporter probe 172 may have a different identification sequence 178 relative to one another but share a same terminal sequence 194.

FIG. 17C shows a model system designed to study splint ligation of reporter probes (e.g., reporter probes 170, 172) in the presence of a ‘MimicMer’, which is a DNA based oligo of similar size to an example aptamer. Two different mimicmers were used with 40% and 50% GC content. The mimicmer oligos and the probes were labelled with Cy3 (green) and the reporters were labelled with Cy5 (red). The probes incubated with T4 DNA ligase for 5, 10, 30 and 60 mins and analyzed by PAGE, as shown in FIG. 17C. Upon successful ligation the largest product is formed at 123 nt. With increasing ligation time the intensity of the largest band increases (as shown by the band intensity plot of FIG. 17D) and the intensity is also highest in the presence of the mimicmers.

FIG. 18A is an embodiment of the technique of FIG. 16 and/or FIG. 17. In particular, use of the splint oligonucleotide 190 can encourage ligation of the reporter probes 170, 172 even without aptamer binding. Exonuclease digestion of free reporter probes 170, 172 can improve background generated from ligation of reporter probes 170, 172 in the absence of aptamer binding. Shown by way of example are exonucleases RecJF and Exo I. Providing a mixture of 5′ to 3′ and 3′ to 5′ exonucleases can encourage sufficient digestion to eliminate or significantly reduce amplification products generated from aptamer-free ligation. FIG. 18B shows a model system designed to study exonuclease protection of splinted ligation of probes in the presence of a ‘MimicMer’ which is a DNA based oligo of similar size to an example aptamer. Two different mimicmers were used that match (are complementary to, bind to) or do not match (e.g., are noncomplementary to, do not bind to) the probe sequences. The mimicmer oligos and the probes were labelled with Cy3 (green) and the H2 oligos are labelled with Cy5 (red). Oligos were incubated with T4 DNA ligase for 30 mins and then subjected to various exonuclease treatments before being analyzed by PAGE. Upon treatment with ExoI, RecJF, or a mixture, the full length product was protected (lane 6, 9, 12) only when ligated in the presence of the correct matching mimicmer. The products of the exonuclease digestion are shown at the side of the gel, as the exonucleases stall at the fluorophores as shown.

FIG. 19 illustrates a workflow in which aptamer-bound reporter probes 170, 172 can be fully circularized form protection from the exonuclease digestion shown in FIG. 15. In particular, the exonuclease digestion targets reporter probes 170, 172 that are not bound to aptamers 14 but that have ligated to one another, e.g., in the presence of the splint oligonucleotide 190.

In certain embodiments, the reporter probes and resultant ligation, extension, or amplification products as discussed herein, e.g., as in FIGS. 16-19, may be used without a capture step.

FIG. 20A shows an example dummy reporter probe technique. In FIG. 20A, a tri-molecular complex 200 is captured using the capture probe 28 via interaction of the bead 36 with the affinity tag 30. The tri-molecular structure includes an associated reporter probe 24 that includes an aptamer binding region 62 and an active or amplifiable nonhybridizing region 64 in which the identification sequence 68 is flanked by primer regions 70, 72. Here, instead of (or in addition to) use of capture probes 28 mixed with dummy probes 32, the reporter probes 24 may also include a mix of active probes 202 and dummy probes 210. Accordingly, other tri-molecular structures may be formed that are associated with an inactive dummy reporter 210. These inactive dummy reporters 210 include the aptamer binding region 62 to facilitate binding to the aptamer 14. However, the nonamplifiable nonhybridizing region 64 of these inactive dummy reporters 210 is not amplifiable. Examples of arrangement of inactive dummy reporters 210 may include a lack of one or both of the primer regions 70, 72, or the identification sequence 68. In another example, the nonamplifiable nonhybridizing region 64 may include an extension blocker, such as an abasic extension blocker, a spacer, or an uracil. In another variant examples, a non-phosphorylated probe can be added to modulate the dynamic range by providing as a mixture including both a version that includes a 5′ phosphate and a version, having a same sequence and aptamer binding capability, but without the available 5′ phosphate. The ratio of the versions may be tuned based on aptamer abundance.

The mix or relative rations of active reporter 202 to inactive dummy reporters 210 may be as generally discussed with respect to capture probe mixtures.

FIG. 20B shows a system designed to confirm dynamic range compression in the presence of dummy biotin or dummy reporters in a trimolecular assay as provided herein. The dummy biotin approach titrated biotin H1 into the Trimolecular assay at 100%, 50%, 20%, 10% and 1%. The dummy reporter used 100%, 50%, 20%, 10% and 1% of a reporter that contained amplification primers that are not amplifiable (M13). Three different MimicMers were used having three different GC contents (50, 60 and 80), and with three different corresponding probe sets for all conditions. Following hybridization, capture, and wash, the libraries were PCR amplified and sequenced on an Illumina sequencer before analyzing and normalizing for counts. Results show that both dummy biotin ‘Dbio’ and dummy reporters ‘Drep’ worked to reduce the read counts for all three mimicmers tested.

FIG. 21 shows reporter probes (e.g., probes 24) with a mix of integral restriction endonuclease (RE) sites located with a nonhybridizing region 64. For example, for a low abundancy aptamer 14, the group 222 of probes 24 may be all the same, e.g., may have no RE site within the nonhybridizing region 64, and instead having a “null” region of nucleotides that does not correspond to the RE site. For a medium abundancy aptamer 14, the group 224 of probes 24 may have a mix of 50% of the probes have the RE site within the nonhybridizing region 64 and 50% not having the RE site, and instead having the null region of nucleotides that does not correspond to the RE site. For a high abundancy aptamer 14, the group 226 of probes 24 may have a mix of 75% of the probes have the RE site within the nonhybridizing region 64 and 25% not having the RE site, and instead having the null region of nucleotides that does not correspond to the RE site. It should be understood that these percentages are by way of example.

The presence of the RE site facilitates cleavage using the appropriate RE. The RE site can be conserved across all aptamers 14 such that only a single RE treatment is required to cleave the nonhybridizing region 64. The cleavage site may be specific for ss DNA cleavage. In such embodiments, the cleavage may occur after capture with the capture probe 28 and before amplification. In other embodiments, the cleavage may occur after amplification using a double-stranded RE. In such cases, the RE site is retained during amplification. The cleaved probes 24 are, thus, unavailable for downstream sequencing and, therefore achieve the dynamic range compression by not being sequenced after amplification. In an embodiment, the null region can differ from the RE site by only a single base substitution to minimize amplification bias between dummy (with RE site) and active (null site, no RE site) probes.

FIG. 22 shows an alternate example that may be used in conjunction with a single probe workflow and/or a double-probe workflow to remove a capture and/or wash step. That is, rather than a tri-molecular complex in which both a capture probe 28 and a reporter probe 24 are used, the illustrated embodiment may be performed using only a reporter probe 24 as generally discussed. Free reporter probes 24 can be removed or digested with exonuclease. Bound reporter probes that are part of a double-stranded complex with the aptamer 14 are protected. However, in certain embodiments, the disclosed exonuclease digestion can be performed in conjunction with other disclosed embodiments, such as with a double-probe workflow using dummy capture probes 32 and/or dummy reporter probes 24 as generally discussed herein. The illustrated embodiment shows 3′ to 5′ exonuclease digestion of free reporters with the 3′ end of the reporter probe 24 being involved in aptamer binding and, therefore, protected from 3′ to 5′exonuclease digestion. The disclosed embodiment may additionally or alternatively be used in conjunction with an exonuclease with 5′ to 3′ exonuclease activity. In such an embodiment, the reporter probe 24 can be designed with the 5′ end being the end that hybridizes to the aptamer 14 to protect the 5′ end from digestion relative to unhybridized reporter probes 24. In certain embodiments, exonuclease digestion may permit workflows with a reduced number of washes and/or improved sensitivity.

FIG. 23 shows an embodiment of bead-based capture using group-specific capture sequences and corresponding different capture bead sets to compress dynamic range for an input library 250 of captured reporter probes 24 or amplified or ligation-extension oligonucleotide products generated from capture reporter probes 24. In one embodiment, the input library 250 represents a population of oligonucleotides 252 having certain universal or common sequences (e.g., adapter sequences 254, 256) shared among the input library 250, certain identification sequences 68 that are unique to only some members of the input library 250 that bind to a particular aptamer 14, and also group-specific capture sequences (e.g., group capture sequences 260, 262, 264) that are different between different groups. The different groups are shown by way of example as a high abundancy group 270, a medium abundancy group 272, and a low abundancy group 274, but more or fewer groups are also contemplated. The estimated abundance of aptamers 14 of a particular aptamer-based assay can be used to divide the aptamers 14 into groups based on relative abundance of the aptamers 14. Once divided, reporter probes 24 designed to bind to aptamers 14 within each group (e.g., groups 270, 272, 274) can include the respective common group capture sequence associated with the abundancy of the group. Any products generated using the reporter probes 24 include the appropriate group capture sequence. Further, in certain embodiment, if the oligonucleotides 252 are products generated using the reporter probes 24, the oligonucleotides 252 may exclude an aptamer binding region (e.g., the second complementary region 62, see FIG. 5), which can be present in the reporter probes 24 but not amplified or included in the input library 250.

The oligonucleotides 252 of a relatively high abundance group 270 may all include a same group capture sequence 260 associated with the high abundancy group 270. It should be understood that, in cases where the oligonucleotides 252 are double-stranded, the oligonucleotides 252 of a relatively high abundancy group 270 may all include either the same group capture sequence 260 or a reverse complement of the group capture sequence 260. Similarly, if the oligonucleotides 252 are double-stranded, the oligonucleotides 252 of all three groups may all include either the universal adapter sequences 256, 258 or reverse complements thereof. As illustrated, a mix of different identification sequences 68 may be present within each group such that the group 270 includes different identification sequences 68a, 68b, 68c that correspond to different aptamers 14a, 14b, 14c that are designated as high abundance. Similarly, the group 272 includes different identification sequences 68d, 68e, 68f that correspond to different aptamers 14d, 14e, 14f that are designated as medium abundance. The low abundance group 274 may also include a mix of different identification sequences 68. In an embodiment, a particular identification sequence 68 is assigned to only one group, such that the identification sequence 68a is only present in the high abundancy group 270 and is only associated with the group capture sequence 260.

After performing the aptamer-based assay and generating the input library 250 from reporter probes 24 bound to aptamers 14 with positive binding events for components of the sample as generally discussed herein, the input library 250 is contacted with different beads 280 of a bead pool 290. The bead pool 290 can include different bead groups 300, 302, 304 with respective different complement regions 310, 312, 314 that are complementary to the bead capture sequences 260, 262, 264. Thus, the oligonucleotides 252 of the high abundancy group 270 that include the bead capture sequence 260 are captured by hybridization to a single-stranded complement region 310 present only in a first bead group 300. Oligonucleotides 252 of the medium abundancy group 272 that include the bead capture sequence 262 are captured by a complement region 312 present only in a second bead group 302, and oligonucleotides 252 of the low abundancy group 274 that include the bead capture sequence 264 are captured by a complement region 314 present only in a second bead group 304. As noted, where the oligonucleotides are double-stranded, only one strand may include the relevant bead capture sequence. Thus, capture may occur after denaturing the oligonucleotides 252 to permit binding to single-stranded complement regions. Once bound, the beads 280 including captured oligonucleotides 252 can be detected as discussed herein. In embodiments, the beads 280 can be designed to generally capture a same amount of oligonucleotides per bead 280 such that each bead group captures about a same amount. However, in certain embodiments, the capture amount per bead 280 for a particular bead group or the number of beads per group may be adjusted to further adjust the concentration of captured oligonucleotides 252 associated with particular aptamers 14.

Different group capture sequences can be incorporated into each reporter probe 24 to permit bead-based capture via hybridization to complementary regions immobilized on the beads 280. If, in contrast, a single common bead capture sequence were used for the entire input library 250, the high abundancy group 270 would tend to be captured in greater proportion on the available beads 280 based on the relatively greater proportion of the oligonucleotides 252 of the high abundancy group 270 within the library 250. By using separate sets of beads 280, dynamic range compression between low abundancy and high abundancy can be achieved. While three separate abundancy groups with corresponding bead groups are illustrated, it should be understood that more or fewer groups are contemplated. In addition, the number of different aptamers 14 and associated identification sequences 68 assigned to each individual group capture sequence may be selected to be one, two, three, ten, 100, 500, or more. In an embodiment, the number of identification sequences 68 assigned to each group may be different. For example, the high abundancy group 270 may include fewer different identification sequences relative to the medium 272 or low abundancy group 274. In addition, the illustrated embodiment may be used alone or in combination with other dynamic range compression techniques as discussed herein (e.g., dummy probes) that may be used to adjust relative abundancies of oligonucleotides 252 of the input library 250. Further, while the workflow is discussed in the context of beads, the capture techniques may be used with surfaces such as flow cells or other substrate.

FIG. 24 shows an example streamlined workflow using direct index amplification, according to an embodiment. In the example workflow, an amplification reaction, e.g., a step out amplification or a direct amplification, can be used to eliminate separate ligation preparation workflow steps. In the left side of the workflow, the captured reporter probe 24 can undergo an amplification reaction that then feeds into a sequence library preparation in which forked adapters are ligated onto ends of the amplified reporter probes. However, amplification to incorporate the sequencing adapter sequences can be used to yield the same end product, but without an intervening ligation step. Thus, the direct amplification workflow, without a ligation step or without the ligation of adapters, can save library preparation time. FIG. 25 is a plot comparing sequencing read counts from the streamlined workflow of FIG. 24 versus a ligation preparation workflow and showing similar sequence read counts, indicating a similar efficiency in library preparation.

FIG. 26 shows an example workflow with wash steps, according to an embodiment. At a first step of the workflow, aptamers 14 are contacted with capture probes 28 and reporter probes 24. The reaction may include a mixture of dummy and non-dummy probes capture probes 28 as disclosed herein. For example, the hybridization reaction to permit aptamer to reporter probe hybridization may be an overnight hybridization by way of example. However, other time ranges are also contemplated (e.g., 30 minutes, 1 hour, 2 hours, 5 hours). If an aptamer 14 that is part of an aptamer-based assay is present in the sample, an aptamer complex is formed that includes the aptamer 14, the capture probe 28, and the reporter probe 24. The probe and aptamer complexes are separated from unbound elements in the reaction mixture via affinity tag capture, illustrated as bead capture. The capture beads include an affinity tag binder such that a capture bead may capture at least one capture probe 28 having an affinity tag. As discussed herein, the beads may also capture empty or uncomplexed probes that are not hybridized to any aptamer. However, uncomplexed capture probes 28, not complexed with a reporter probe 24 via an aptamer, will not yield any amplification products at downstream steps.

Once captured on beads, a wash step is performed to separate the beads from unbound elements, which include reporter probes 24 that are not complexed with any aptamer as well as dummy complexes that may include reporter probes 24 complexed with a dummy probe with no affinity tag. After the separation, the sample proceeds to sequence library preparation steps, shown as a ligation to PCR reaction. However, other preparation workflows are also contemplated, such as direct amplification, step out PCR, or other amplification and/or ligation preparations as discussed herein. The end products of the workflow include oligonucleotide fragments that can then be sequenced as part of a sequencing reaction to generate sequence data.

In an embodiment, the workflow can include only a single wash step after bead capture and before amplification and/or ligation steps. In other embodiments, two, three or more wash steps are contemplated. FIG. 27 shows sequencing read counts for different wash conditions to wash at the bead capture step and comparing 3, 6, and 12 washes. Reducing a number of washing steps from 12 to 6 improves reproducibility and reduces assay time and use of consumables. Reducing washes further from 6 to 3 further increases the signal, but background also increases without any input (0 input fM).

FIG. 28A shows compression of sequencing read counts using a dummy-biotin for different aptamers. The left side panel shows an experimental setup with aptamer and probe complex formation. Two different types of complexes may be formed for an individual aptamer: a first complex that includes an affinity tag and a second complex that does not include an affinity tag. The ratio of these types of complexes for a given aptamer is dependent on a ratio of dummy probes to capture probes. FIG. 28A shows that sequence read counts are reduced via the use of dummy probes in the workflow of FIG. 26 to remove part of the aptamer population that, if not removed, would have generated sequence reads. FIG. 28A shows a reduced readcount by 2 orders of magnitude (100×), i.e. compression to 1% across a panel of 96 aptamers. FIG. 28B shows results from a trimolecular NGS conversion assay performed using probes targeting an aptamer panel. 20 uL of assay eluate from pooled human plasma sample (10 donors) was added to the trimolecular conversion assay. For the ‘POS’ control, all of the probes had 100% biotin. For the ‘DRC’ sample (Dynamic Range Compression), the probes were split into 4 virtual groups using different % of biotin. The amount of biotin for the four virtual groups was 0.065%, 0.63%, 5.42% and 100%. Samples were sequenced on NovaSeq 6000 and data were normalized for counts analysis (y-axis) for each of the aptamers (SeqID-x-axis).

FIG. 29 shows example undesired nonspecific binding between aptamer binding regions. The top of FIG. 29 shows a desired complex structure after a hybridization reaction in which the complex includes the aptamer 14, the reporter probe 24, and the capture probe 28. The bottom of FIG. 29 shows undesired structure formation in which the reporter probe 24 complexes directly with the capture probe 28 via the aptamer binding region of the reporter probe 24 and/or the aptamer binding region of the capture probe 28. Here, the complex is formed without any aptamer bridge. Pulling the undesired reporter probe 24 down during bead capture and subsequent amplification and sequencing results in background due to nonspecific binding. FIG. 30 shows contributions of different aptamer binding regions to non-specific binding. Non-specific aptamer binding region interactions were shown to be a main contributor to background. The non-specific binding may be low level base-paring between adaptor sequences.

FIG. 31 is a schematic diagram of a sequencing device 500 that may be used in conjunction with the disclosed embodiments for acquiring sequencing data of identification sequences and/or index sequences as generally discussed herein. The sequence device 500 may be implemented according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device 500. Such techniques use DNA ligase to incorporate oligonucleotides and identify the incorporation of such oligonucleotides and are described in U.S. Pat. Nos. 6,969,488; 6,172,218; and 6,306,597; the disclosures of which are incorporated herein by reference in their entireties. Some embodiments can utilize nanopore sequencing, whereby target nucleic acid strands, or nucleotides exonucleolytically removed from target nucleic acids, pass through a nanopore. As the target nucleic acids or nucleotides pass through the nanopore, each type of base can be identified by measuring fluctuations in the electrical conductance of the pore (U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties). Yet other embodiments include detection of a proton released upon incorporation of a nucleotide into an extension product. For example, sequencing based on detection of released protons can use an electrical detector and associated techniques that are commercially available from Ion Torrent (Guilford, C T, a Life Technologies subsidiary) or sequencing methods and systems described in US 2009/0026082 A1; US 2009/0127589 A1; US 2010/0137143 A1; or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides, or with zeromode waveguides as described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, the sequencing device 500 may be a HiSeq, MiSeq, or HiScanSQ from Illumina (La Jolla, CA). In other embodiment, the sequencing device 500 may be configured to operate using a CMOS sensor with nanowells fabricated over photodiodes such that DNA deposition is aligned one-to-one with each photodiode.

The sequencing device 500 may be “one-channel” a detection device, in which only two of four nucleotides are labeled and detectable for any given image. For example, thymine may have a permanent fluorescent label, while adenine uses the same fluorescent label in a detachable form. Guanine may be permanently dark, and cytosine may be initially dark but capable of having a label added during the cycle. Accordingly, each cycle may involve an initial image and a second image in which dye is cleaved from any adenines and added to any cytosines such that only thymine and adenine are detectable in the initial image but only thymine and cytosine are detectable in the second image. Any base that is dark through both images in guanine and any base that is detectable through both images is thymine. A base that is detectable in the first image but not the second is adenine, and a base that is not detectable in the first image but detectable in the second image is cytosine. By combining the information from the initial image and the second image, all four bases are able to be discriminated using one channel.

In the depicted embodiment, the sequencing device 500 includes a separate sample processing device 502 and an associated computer 504. However, as noted, these may be implemented as a single device. Further, the associated computer 504 may be local to or networked or otherwise in communication with the sample processing device 502. In the depicted embodiment, the biological sample may be loaded into the sample processing device 502 on a sample substrate 510, e.g., a flow cell or slide, that is imaged to generate sequence data. For example, reagents that interact with the biological sample fluoresce at particular wavelengths in response to an excitation beam generated by an imager 512 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imager 512.

The imager detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imager 512 may be under processor control, e.g., via a processor 514, and the sample receiving device 502 may also include I/O controls 516, an internal bus 518, non-volatile memory 520, RAM 522 and any other memory structure such that the memory is capable of storing executable instructions, and other suitable hardware components that may be similar to those described with regard to FIG. 31. Further, the associated computer 504 may also include a processor 524, I/O controls 526, communications circuitry 527, and a memory architecture including RAM 528 and non-volatile memory 530, such that the memory architecture is capable of storing executable instructions 532. The hardware components may be linked by an internal bus, which may also link to the display 534. In embodiments in which the sequencing device 500 is implemented as an all-in-one device, certain redundant hardware elements may be eliminated.

The processor 514, 524 may be programmed to assign individual sequencing reads to a sample based on the associated index sequence or sequences according to the techniques provided herein. In particular embodiments, based on the image data acquired by the imager 512, the sequencing device 500 may be configured to generate sequencing data that includes base calls for each base of a sequencing read. Further, based on the image data, even for sequencing reads that are performed in series, the individual reads may be linked to the same location via the image data and, therefore, to the same template strand. In this manner, index sequencing reads may be associated with a sequencing read of an insert sequence before being assigned to a sample of origin. The processor 514, 524 may also be programmed to perform downstream analysis on the sequences corresponding to the inserts for a particular sample subsequent to assignment of sequencing reads to the sample.

In certain embodiments, the I/O controls 516, 526 may be configured to receive user inputs that automatically select sequencing parameters based on the reporter probes 24 and the associated sequence library preparation techniques. For example, in cases where custom primers or dark cycles are incorporated into the sequencing run, the sequencing device can select from preprogrammed operating instructions and/or receive user inputs to cause the sequencing device to operate according to the desired sequence parameters. In an embodiment, the user input may be a selection of a sequence library preparation kit or reading a barcode or identifier of a sequence library preparation kit.

In embodiments of the disclosed techniques, aptamer detection may be based on a presence of the uniquely identifying identification sequence 68 for an individual aptamer in sequencing data generated by the sequencing device 500. Accordingly, in an embodiment, the sequencing device 500 may perform analysis of sequence reads to identify one or more identification sequences 68 for a panel of aptamers. Based on the identified aptamers, a notification or report of positive aptamer identification may be generated. In an embodiment, the notification is provided on the display 534 or communicated via the communications circuitry 527 to a remote device or a cloud server.

As used herein, an aptamer may refer to a non-naturally occurring nucleic acid that has specific binding affinity for a target molecule. The binding of the aptamer to the target molecule can result in catalytically changing the target molecule, reacting with the target molecule in a way that modifies or alters the target molecule or the functional activity of the target molecule, covalently attaching to the target molecule (as in a suicide inhibitor), and facilitating the reaction between the target molecule and another molecule. In one embodiment, the target molecule is a three dimensional chemical structure, other than a polynucleotide, that binds to the aptamer through a mechanism which is predominantly independent of Watson/Crick base pairing or triple helix binding. In an embodiment, the aptamer is not a nucleic acid having the known physiological function of being bound by the target molecule.

Aptamers include nucleic acids that are identified from a candidate mixture of nucleic acids. A specific binding affinity of an aptamer for its target may refer to aptamer binding to its target generally with a much higher degree of affinity than it binds to other, non-target, components in a mixture or sample. Different aptamers may have either the same number or a different number of nucleotides. Aptamers may be DNA or RNA and may be single stranded, double stranded, or contain double stranded regions. The aptamers discussed herein can be used in any diagnostic, imaging, high throughput screening or target validation techniques or procedures or assays for which aptamers, oligonucleotides, antibodies and ligands, without limitation can be used.

Aptamers as disclosed herein may be used in aptamer-based assays, such as those disclosed in U.S. Pat. Nos. 7,855,054 and 7,964,356 and U.S. Publication Nos. US/2011/0136099 and US/2012/0115752. In one example, a panel of aptamers to different target molecules is provided attached to a solid support. The attachment of the aptamers to the solid support is accomplished by contacting a first solid support with the aptamer/s and allowing the releasable first tag included on the aptamer to associate, either directly or indirectly, with an appropriate first capture agent that is attached to or part of the first solid support. A test sample is then prepared and contacted with the immobilized aptamers that have a specific affinity for their respective target molecules, which may or may not be present in the sample. If the test sample contains the target molecule(s), an aptamer-target affinity complex will form in the mixture with the test sample. In addition to aptamer-target affinity complexes, uncomplexed aptamer will also be attached to the first solid support. The aptamer-target affinity complex and uncomplexed aptamer that has associated with the probe on the solid support is then partitioned from the remainder of the mixture, thereby removing free target and all other uncomplexed matter in the test sample (sample matrix); i.e., components of the mixture not associated with the first solid support. This partitioning step is referred to herein as the Catch-1 partition (see definition below). Following partitioning the aptamer-target affinity complex, along with any uncomplexed aptamer, is released from the first solid support using a method appropriate to the particular releasable first tag being employed.

In one embodiment, aptamer-target affinity complexes bound to the solid support are treated with an agent that introduces a second tag to the target molecule component of the aptamer-target affinity complexes. In one embodiment, the target is a protein or a peptide, and the target is biotinylated by treating it with NHS-PEO4-biotin. The second tag introduced to the target molecule may be the same as or different from the aptamer capture tag. If the second tag is the same as the first tag, or the aptamer capture tag, free capture sites on the first solid support may be blocked prior to the initiation of this tagging step. In this exemplary embodiment, the first solid support is washed with free biotin prior to the initiation of target tagging. Tagging methods, and in particular, tagging of targets such as peptides and proteins are described in U.S. Pat. No. 7,855,054.

Partitioning is completed by releasing of uncomplexed aptamers and aptamer-target affinity complexes from the first solid support. In one embodiment, the first releasable tag is a photocleavable moiety that is cleaved by irradiation with a UV lamp under conditions that cleave ≥90% of the first releasable tag. In other embodiments, the release is accomplished by the method appropriate for the selected releasable moiety in the first releasable tag. Aptamer-target affinity complexes may be eluted and collected for further use in the assay or may be contacted to another solid support to conduct the remaining steps of the assay.

In one embodiment, a second partition is performed (referred to herein as the Catch-2 partition, see definition below) to remove free aptamer. As described above, in one embodiment, a second tag used in the Catch-2 partition may be added to the target while the aptamer-target affinity complex is still in contact with the solid support used in the Catch-0 capture. In other embodiments, the second tag may be added to the target at another point in the assay prior to initiation of Catch-2 partitioning. The mixture is contacted with a solid support, the solid support having a capture element (second) adhered to its surface which is capable of binding to the target capture tag (second tag), preferably with high affinity and specificity. In one embodiment, the solid support is magnetic beads (such as DynaBeads MyOne Streptavidin C1) contained within a well of a microtiter plate and the capture element (second capture element) is streptavidin. The magnetic beads provide a convenient method for the separation of partitioned components of the mixture. Aptamer-target affinity complexes contained in the mixture are thereby bound to the solid support through the binding interaction of the target (second) capture tag and the second capture element on the second solid support. The aptamer-target affinity complex is then partitioned from the remainder of the mixture, e.g. by washing the support with buffered solutions, including buffers comprising organic solvents including, but not limited to glycerol.

Aptamers are then selectively eluted from aptamer-target complexes with buffers comprising chaotropic salts from the group including, but not limited to sodium perchlorate, lithium chloride, sodium chloride and magnesium chloride. Aptamers retained on Catch-2 beads by virtue of aptamer/aptamer interaction are not eluted by this treatment.

In another embodiment, the aptamer released from the Catch-2 partition is detected and optionally quantified by detection methods as discussed herein, such as via next generation sequencing techniques. For example, via amplification and/or sequencing of probes that bind to the eluted aptamers. In certain embodiments, the detection includes detection results that provide relative and/or estimated absolute concentrations of detected aptamers. The detection results may include a notification or output of a positive or negative detection result or a relative concentration or estimated concentration for a particular aptamer ID or a particular target of the aptamer.

In certain embodiments of the disclosure, the disclosed probes of the probe set 20 can include one or more conserved regions, such as a conserved primer region, e.g., a first conserved primer region and a second conserved primer region. A conserved region is conserved between at least some other probes of the probe set 20 such that the conserved region has an identical or similar nucleotide sequence as compared between the probes. For example, for a given second probe 24, all probes 24 can have a same first conserved primer region and a second conserved primer region. In this manner, primers based on the first conserved primer region and the second conserved primer region can be used to amplify any captured probes 24.

One or more probes as discussed herein may include an identification sequence that can include one or more nucleotide sequences that can be used to identify one or more specific aptamers. The identification sequence can be an artificial sequence. The identification sequence can comprise at least about 1, 2, 3, 4, 5, 6, 7, 8, 9, 10, 11, 12, 13, 14, 15, 16, 17, 18, 19, 20 or more consecutive nucleotides. In some embodiments, the identification sequence comprises at least about 10, 20, 30, 40, 50, 60, 70 80, 90, 100 or more consecutive nucleotides. In some embodiments, at least a portion of the identification sequence in a probes is different.

One or more probes as discussed herein may include an affinity tag. Affinity tags can be useful for a variety of applications, for example the bulk separation of target nucleic acids hybridized to hybridization tags. As used herein, the term “affinity tag” and grammatical equivalents can refer to a component of a multi-component complex, wherein the components of the multi-component complex specifically interact with or bind to each other. For example an affinity tag can include biotin or poly-His that can bind streptavidin or nickel, respectively. Other examples of multiple-component affinity tag complexes are listed, for example, U.S. Patent Application Pub. No. 2012/0208705, U.S. Patent Application Pub. No. 2012/0208724 and Int. Patent Application Pub. No. WO 2012/061832, each of which is incorporated by reference in its entirety.

The disclosed embodiments provide a different primers and probes. Probes and/or primers of the disclosed embodiments are designed to be complementary to a target sequence (either the target sequence of the sample or to other probe sequences), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions. Longer sequences hybridize specifically at higher temperatures. Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides).

In certain embodiments, probe contacting steps may be run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc. The size of the primer nucleic acid may vary, as will be appreciated by those in the art, in general varying from 5 to 500 nucleotides in length. Primers may be between 10 and 100, between 15 and 50, and from 10 to 35 depending on the use and amplification technique.

The disclosed techniques are directed to dynamic range compression in one or more applications, such as for analysis of an eluate of an aptamer-based assay. The dynamic range compression may include one or more amplification steps that can be part of sequencing library preparation that may oligonucleotide adapters to reporter probes for downstream sequencing. The adapters may be attached to the target polynucleotide in any other suitable manner. In some embodiments, the adapters are introduced in a multi-step process, such as a two-step process, involving ligation of a portion of the adapter to the target polynucleotide having a universal primer sequence. The second step includes extension, for example by PCR amplification, using primers that include a 3′ end having a sequence complementary to the attached universal primer sequence and a 5′ end that contains other sequences of an adapter. By way of example, such extension may be performed as described in U.S. Pat. No. 8,053,192, which is hereby incorporated by reference in its entirety. Additional extensions may be performed to provide additional sequences to the 5′ end of the resulting previously extended polynucleotide.

In some embodiments, the adapter may be ligated to the reporter probes. Any suitable adapter may be attached to a target polynucleotide, such as a reporter probe, via any suitable process, such as those discussed herein. The adapter can include a library-specific index tag sequence (e.g., i5, i7). The index tag sequence may be attached to the target polynucleotides from each library before the sample is immobilized for sequencing. The index tag is not itself formed by part of the target polynucleotide, but becomes part of the template for amplification. The index tag may be a synthetic sequence of nucleotides which is added to the target as part of the template preparation step. Accordingly, a library-specific index tag is a nucleic acid sequence tag which is attached to each of the target molecules of a particular library, the presence of which is indicative of or is used to identify the library from which the target molecules were isolated. Preferably, the index tag sequence is 20 nucleotides or less in length. For example, the index tag sequence may be 1-10 nucleotides or 4-6 nucleotides in length. A four nucleotide index tag gives a possibility of multiplexing 256 samples on the same array, a six base index tag enables 4,096 samples to be processed on the same array. The adapters may contain more than one index tag so that the multiplexing possibilities may be increased.

The adapters may include any other suitable sequence in addition to the index tag sequence. For example, the adapters may include universal extension primer sequences, which are typically located at the 5′ or 3′ end of the adapter and the resulting polynucleotide for sequencing. The universal extension primer sequences may hybridize to complementary primers bound to a surface of a solid substrate. The complementary primers include a free 3′ end from which a polymerase or other suitable enzyme may add nucleotides to extend the sequence using the hybridized library polynucleotide as a template, resulting in a reverse strand of the library polynucleotide being coupled to the solid surface. Such extension may be part of a sequencing run or cluster amplification.

In some embodiments, the adapters include one or more universal sequencing primer sequences. The universal sequencing primer sequences may bind to sequencing primers to allow sequencing of an index tag sequence, a target sequence, or an index tag sequence and a target sequence. In some embodiments, the disclosed reporter probes, e.g., reporter probe 24, may include a “sequencing adaptor” or “sequencing adaptor site”, that is to say a region that comprises one or more sites that can hybridize to a primer. In some embodiments, a sequence can include at least a first primer site useful for amplification, sequencing, and the like.

After adapter incorporation, the disclosed reporter probes may be sequenced. In one example, the sequencing may be via Illumina's sequencing-by-synthesis and reversible terminator-based sequencing chemistry. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchors. Under limiting-dilution conditions, adapter-modified, single-stranded template DNA is added to the flow cell and immobilized by hybridization to the anchors. Attached DNA fragments are extended and bridge amplified to create an ultra-high density sequencing flow cell with hundreds of millions of clusters, each containing ˜1,000 copies of the same template. In one embodiment, the randomly fragmented genomic DNA is amplified using PCR before it is subjected to cluster amplification. Alternatively, an amplification-free genomic library preparation is used, and the randomly fragmented genomic DNA is enriched using the cluster amplification alone. The templates are sequenced using a robust four-color DNA sequencing-by-synthesis technology that employs reversible terminators with removable fluorescent dyes. High-sensitivity fluorescence detection is achieved using laser excitation and total internal reflection optics. Sequence are aligned against a truth table or stored correlations between aptamer identity and identification sequences using specially developed data analysis pipeline software.

This written description uses examples to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
contacting the individual aptamer with a mixture of first probes, wherein a first complementary region of each first probe of the mixture hybridizes to a first region of the individual aptamer and wherein only a subset of the first probes in the mixture are coupled to an affinity tag;
contacting the individual aptamer with a second probe to hybridize a second complementary region of the second probe to a second region of the individual aptamer and wherein the second probe comprises a nonhybridizing region extending from the complementary region, the nonhybridizing region comprising an identification sequence uniquely identifying for the individual aptamer, wherein the first complementary region and the second complementary region uniquely hybridize to the individual aptamer;
capturing a first probe of the mixture via binding of the affinity tag to an affinity tag binder to capture the individual aptamer and the second probe hybridized to the second region of the individual aptamer, wherein the first probe is in the subset coupled to the affinity tag; and
detecting the identification sequence of the captured second probe.

2. The method of claim 1, wherein the first region and the second region are spaced apart by at least one nucleotide.

3. The method of claim 1, wherein the affinity tag is biotin and the affinity tag capture molecule is avidin or streptavidin.

4. The method of claim 1, wherein detecting the identification sequence of the captured second probe comprises contacting the captured second probe with primers to generate an amplification product.

5. The method of claim 4, wherein the primers comprise a first primer that binds to a first primer binding region of the nonhybridizing region and a second primer that binds to a second primer binding region of the nonhybridizing region, wherein the first primer binding region and the second primer binding region flank the identification sequence.

6. The method of claim 5, wherein the first primer comprises a first sequencing primer and the second primer comprises a second sequencing primer such that the amplification product comprises the first sequencing primer and the second sequencing primer.

7. The method of claim 4, wherein detecting the identification sequence of the captured second probe comprises sequencing the amplification product.

8. The method of claim 1, further comprising generating a notification related to the individual aptamer based on detecting the identification sequence.

9. The method of claim 1, further comprising separating the captured second probe from the captured first probe and the captured individual aptamer before detecting the identification sequence.

10. The method of claim 1, further comprising ligating an oligonucleotide to an end of the captured second probe and extending the ligated oligonucleotide before detecting the identification sequence.

11. The method of claim 1, further comprising hybridizing a first oligonucleotide and a second oligonucleotide to the captured second probe and extending the first oligonucleotide using the captured second probe as a template to ligate the first oligonucleotide to the second oligonucleotide via extension ligation.

12. The method of claim 11, further comprising separating the ligated first oligonucleotide and second oligonucleotide from the captured second probe using a second affinity tag coupled to the second oligonucleotide.

13. The method of claim 12, further comprising hybridizing a third oligonucleotide to the ligated first oligonucleotide and second oligonucleotide and extending the ligated first oligonucleotide and second oligonucleotide and the hybridized third oligonucleotide to generate first and second complementary strands comprising 5′ and 3′ adapters for sequencing.

14. The method of claim 1, further comprising cleaving at least a portion of the nonhybridizing region comprising the identification sequence from the captured second probe before detecting the identification sequence.

15. The method of claim 14, further comprising ligating a cleaved portion of the captured second probe to an end of an adapter after the cleaving.

16. The method of claim 1, further comprising removing uncaptured probes before detecting the identification sequence.

17. The method of claim 16, wherein the uncaptured probes comprise probes in the mixture not in the subset, wherein the probes not in the subset are not coupled to the affinity tag.

18. An aptamer detection probe set, comprising:
a plurality of different first probe mixtures complementary to respective different aptamers of an aptamer panel, wherein an individual first probe mixture of the plurality of different first probe mixtures comprises:
a binding subset of first probes coupled to an affinity tag;
a dummy subset of first probes not coupled to the affinity tag, and wherein each probe in the binding subset and the dummy subset of the individual first probe mixture comprises a same binding region that is complementary to a first sequence of an individual aptamer of the aptamer panel; and
a plurality of different second probes complementary to the respective different aptamers of the aptamer panel, wherein an individual second probe of the plurality of different second probes comprises a second binding region complementary to a second sequence of the individual aptamer and wherein the individual second probe comprises a nonhybridizing region, the nonhybridizing region comprising an identification sequence uniquely identifying for the individual aptamer.

19. The probe set of claim 18, wherein the identification sequence is nonoverlapping with a sequence of the individual aptamer.

20. The probe set of claim 18, wherein the identification sequence is 20 nucleotides or fewer in length.

21. The probe set of claim 18, wherein the individual second probe comprises a cleavage site disposed between the nonhybridizing region and the second binding region.

22. The probe set of claim 18, wherein the nonhybridizing region comprises primer regions that flank the identification sequence.

23. The probe set of claim 18, wherein the binding subset comprises less than 20% of the individual first probe mixture.

24. The probe set of claim 18, wherein a ratio of the binding subset to the dummy subset is less than 1:1 in the individual first probe mixture.

25. The probe set of claim 18, comprising a second first probe mixture of the plurality of different comprising:
a second binding subset of first probes coupled to an affinity tag;
a second dummy subset of first probes not coupled to affinity tag, and wherein each probe in the second binding subset and the second dummy subset of the second first probe mixture comprises a same second binding region that is complementary to an second aptamer of the aptamer panel, the second aptamer being different than the individual aptamer and the second binding region being different than the binding region.

26. The probe set of claim 25, wherein a first ratio of the binding subset to the dummy subset in the individual first probe mixture is different than a second ratio of the second binding subset to the second dummy subset in the second first probe mixture.

27. An aptamer detection probe set, comprising:
a plurality of different first probe mixtures complementary to respective different aptamers of an aptamer panel, wherein each first probe mixture of the plurality of different first probe mixtures comprises:
a binding subset of first probes;
a dummy subset of first probes not coupled to affinity tag, and wherein each first probe mixture comprises a binding region that is complementary to an aptamer of the aptamer panel, and wherein the binding region is unique to each first probe mixture such that each first probe mixture binds to a different aptamer of the aptamer panel, wherein each first probe mixture has a different ratio of the binding subset to the dummy subset relative to the other first probe mixtures of the plurality; and
a plurality of different second probes complementary to the respective different aptamers of the aptamer panel.

28. The probe set of claim 27, wherein the plurality of different first probe mixtures comprises at least 100 different first probe mixtures.

29. An aptamer detection probe set, comprising:
a plurality of different reporter probe mixtures complementary to respective different aptamers of an aptamer panel, wherein an individual reporter probe mixture of the plurality of different reporter probe mixtures comprises:
a first subset of reporter probes comprising an amplifiable nonhybridizing region, the nonhybridizing region comprising an identification sequence uniquely identifying for an individual aptamer that is flanked by a first primer region and a second primer region and that is capable of being amplified using primers complementary to or corresponding to the first primer region and the second primer region; and
a second subset of reporter probes, and wherein each probe in the first subset and the second subset of the individual reporter probe mixture comprises a same binding region that is complementary to a first sequence of an individual aptamer of the aptamer panel, wherein the individual second probe comprises a nonamplifiable nonhybridizing region that is not capable of being amplified using the primers; and
a plurality of different capture probes complementary to the respective different aptamers of the aptamer panel, wherein an individual capture probe of the plurality of different capture probes comprises a second binding region complementary to a second sequence of the individual aptamer and, wherein each capture probe of the plurality is coupled to an affinity tag.

30. The probe set of claim 29, wherein the nonamplifiable nonhybridizing region does not comprise one or both of the first primer region and the second primer region.

31. The probe set of claim 29, wherein the nonamplifiable nonhybridizing region does not comprise the identification sequence.

32. The probe set of claim 29, wherein the nonamplifiable nonhybridizing region comprises at least one extension blocker.

33. The probe set of claim 29, wherein the nonamplifiable nonhybridizing region comprises a restriction enzyme site.

34. A method of aptamer detection, comprising:
contacting an individual aptamer with reporter probes that hybridize a first region of the individual aptamer, wherein a first subset of the reporter probes comprise an amplifiable nonhybridizing region, the amplifiable nonhybridizing region comprising an identification sequence uniquely identifying for the individual aptamer that is flanked by a first primer region and a second primer region and a second subset comprise a nonamplifiable nonhybridizing region;
contacting the individual aptamer with a capture probe, wherein the capture probes hybridize to a second region of the individual aptamer and wherein the capture probe is associated with an affinity tag;
capturing the capture probe via binding of the affinity tag to an affinity tag binder to capture the individual aptamer and the reporter probe comprising the amplifiable nonhybridizing region hybridized to the first region of the individual aptamer; and
detecting the identification sequence of the captured reporter probe via amplification of the identification sequence.

35. The method of claim 34, wherein detecting the identification sequence of the captured reporter probe comprises contacting the captured reporter probe with primers to generate an amplification product.

36. The method of claim 35, wherein the primers comprise a first primer that binds to a first primer binding region of the nonhybridizing region and a second primer that corresponds to a second primer binding region of the nonhybridizing region, wherein the first primer binding region and the second primer binding region flank the nonhybridizing region.

37. The method of claim 35, wherein detecting the identification sequence of the captured reporter probe comprises sequencing the amplification product.

38. The method of claim 34, wherein reporter probes comprising the nonamplifiable nonhybridizing region hybridized to the first region of the individual aptamer are not detected.

39. A method of aptamer detection, comprising:
contacting aptamers with probes that hybridize to respective different aptamers of the aptamers, wherein a complementary region of each probe hybridizes to an individual aptamer of the aptamers and wherein each probe comprises a nonhybridizing region extending from the complementary region, the nonhybridizing region comprising an identification sequence uniquely identifying for each individual aptamer;
using an exonuclease to remove excess probes not hybridized to the aptamers; and
detecting the identification sequence in the probes after removing the excess probes.

40. A method of aptamer detection, comprising:
contacting a first aptamer and a second aptamer with reporter probes, the reporter probes comprising a first subset that hybridize to the first aptamer and a second subset that hybridize to the second aptamer, wherein the reporter probes of the first subset comprise a first nonhybridizing region comprising a first identification sequence uniquely identifying for the first aptamer and a first group capture sequence and wherein the reporter probes of the second subset comprise a second nonhybridizing region comprising a second identification sequence uniquely identifying for the second aptamer and the first group capture sequence;
contacting the first aptamer and the second aptamer with capture probes, wherein the capture probes hybridize to the first aptamer or the second aptamer and wherein the capture probes are associated with an affinity tag;
capturing the capture probes via binding of the affinity tag to an affinity tag binder to capture the first aptamer and the second aptamer and the reporter probes;
generating first oligonucleotides comprising the first identification sequence and the first group capture sequence from the first subset and second oligonucleotides comprising the second identification sequence and the first group capture sequence from the second subset;
capturing the first oligonucleotides and the second oligonucleotides using a first group of beads carrying a sequence complementary to the first group capture sequence; and
detecting the first identification sequence in the captured first oligonucleotides and the second identification sequence in the second oligonucleotides to detect the first aptamer and the second aptamer.

41. The method of claim 40, comprising contacting a third aptamer with the reporter probes, the reporter probes comprising a third subset comprise a third nonhybridizing region comprising a third identification sequence uniquely identifying for the third aptamer and a second group capture sequence different than the first group capture sequence.

42. The method of claim 41, comprising:
generating third oligonucleotides comprising the third identification sequence and the second group capture sequence from the third subset;
capturing the third oligonucleotides using a second group of beads carrying a sequence complementary to the second group capture sequence; and
detecting the third identification sequence in the captured third oligonucleotides to detect the third aptamer.

43. The method of claim 42, wherein the first group of beads and the second group of beads comprise about a same number of beads.

44. A method of aptamer detection, comprising:
contacting an individual aptamer with a first reporter probe that hybridizes to a first region of the individual aptamer, wherein the first reporter probe comprises a first nonhybridizing region, the nonhybridizing region comprising a first identification sequence uniquely identifying for the individual aptamer and with a second reporter probe that hybridize to a second region of the individual aptamer, wherein the second reporter probe comprises a second nonhybridizing region, the second nonhybridizing region comprising a second identification sequence uniquely identifying for the individual aptamer;
ligating ends of the first identification sequence and the second identification sequence to one another to generate ligated reporter probes;
capturing ligated reporter probes using an affinity tag coupled to the first reporter probe or the second reporter probe; and
detecting the first identification sequence and the second identification sequence via amplification of the captured ligated reporter probes to detect the individual aptamer.

45. The method of claim 44, comprising hybridizing a single-stranded oligonucleotide splint to the first reporter probe and the second reporter probe.

46. The method of claim 44, comprising using one or more exonucleases to digest other reporter probes not bound to the individual aptamer or other aptamers.

47. The method of claim 44, comprising using ligase to circularize the captured ligated reporter probes.

48. The method of claim 44, wherein the first reporter probe comprises a first primer region and wherein the second reporter probe comprises a second primer region, and wherein the amplification uses primers complementary to or corresponding to the first primer region and the second primer region.

49. The method of claim 48, wherein the first primer region is positioned between the first identification sequence and a sequence that bind to the aptamer on the first reporter probe.

50. The method of claim 49, wherein the second primer region is positioned between the second identification sequence and a sequence that bind to the aptamer on the second reporter probe.

51. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
contacting the individual aptamer with a mixture of first probes, wherein a first complementary region of each first probe of the mixture is capable of hybridizing to a first region of the individual aptamer and wherein only a subset of the first probes in the mixture are coupled to an affinity tag such that a first probe of the mixture hybridizes to the first region of the individual aptamer;
contacting the individual aptamer with a second probe to hybridize a second complementary region of the second probe to a second region of the individual aptamer and wherein the second probe comprises a nonhybridizing region extending from the complementary region, the nonhybridizing region comprising an identification sequence uniquely identifying for the individual aptamer, wherein the first complementary region and the second complementary region uniquely hybridize to the individual aptamer;
ligating the first probe hybridized to the first region of the individual aptamer to the second probe hybridized to the second region of the individual aptamer;
capturing the first probe via binding of the affinity tag to an affinity tag binder to capture the individual aptamer and the second probe hybridized to the second region of the individual aptamer and ligated to the first probe, wherein the first probe is in the subset coupled to the affinity tag; and
detecting the identification sequence of the captured second probe.

52. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
contacting the individual aptamer with a mixture of first probes, wherein a first complementary region of each first probe of the mixture hybridizes to a first region of the individual aptamer and wherein only a subset of the first probes in the mixture are coupled to an affinity tag;
contacting the individual aptamer with a second probe to hybridize a second complementary region of the second probe to a second region of the individual aptamer and wherein the second probe comprises a nonhybridizing region extending from the complementary region, the nonhybridizing region comprising an identification sequence uniquely identifying for the individual aptamer, wherein the first complementary region and the second complementary region uniquely hybridize to the individual aptamer;
capturing a first probe of the mixture via binding of the affinity tag to an affinity tag binder to capture the individual aptamer and the second probe hybridized to the second region of the individual aptamer, wherein the first probe is in the subset coupled to the affinity tag;
generating amplification products from the captured second probe using a primer pair, wherein the primer pair comprises a first primer complementary to a region of the second probe that does not include the second complementary region and that does not include the identification sequence; and
sequencing the amplification products.

53. The method of claim 52, wherein the nonhybridizing region of the second probe comprises a first adapter sequence and a second adapter sequence that flank the identification sequence.

54. The method of claim 53, wherein the primer pair comprises sequences complementary to or comprising portions of the first adapter sequence or the second adapter sequence.

55. The method of claim 53, wherein sequencing the amplification products comprises using sequencing primers complementary to or comprising portions of the first adapter sequence or the second adapter sequence.

56. The method of claim 53, wherein the first adapter sequence comprises a first index sequence and the second adapter sequence comprises a second index sequence.

57. The method of claim 52, comprising separating the captured first probe from the first probes in the mixture not coupled to the affinity tag via a wash step.

58. The method of claim 57, wherein the wash step comprises six or fewer washes.

59. A sequencing method, comprising:
generating sequence data from a sequence library, wherein the sequence library is prepared by:
contacting analytes of a sample with a plurality of aptamers under conditions that permit analyte-aptamer complexes to form, wherein different aptamers of the plurality of aptamers have specific affinity for respective different analytes of the analytes;
forming a first plurality of aptamer complexes of a first type by hybridizing a reporter probe and a dummy probe to an individual aptamer of the plurality of aptamers, wherein the dummy probe comprises a first complementary region that hybridizes to a first region of the individual aptamer and wherein the reporter probe comprises a second complementary region that hybridizes to a second region of the individual aptamer and a nonhybridizing region extending from the complementary region, the nonhybridizing region comprising an identification sequence uniquely identifying for the individual aptamer;
forming a second plurality of aptamer complexes of a second type by hybridizing the reporter probe and a capture probe to the individual aptamer, wherein the capture probe comprises the first complementary region that hybridizes to a first region of the individual aptamer and an affinity tag;
separating the second plurality of aptamer complexes from the first plurality via the affinity tag to generate a separated second plurality of aptamer complexes; and
amplifying a portion of the reporter probes of the separated
second plurality of aptamer complexes to generate the sequence library;
identifying the identification sequence in the sequence data; and
generating a notification that the individual aptamer is present in the sample based on the identifying.

60. The method of claim 59, wherein the notification is displayed on a display of a sequencing device or a computer in communication with the sequencing device.

61. The method of claim 59, wherein generating sequence data from a sequence library using sequencing primers complementary to or comprising portions of the amplified portions of the reporter probes.

62. The method of claim 59, wherein the sequence library is formed by forming additional pluralities of aptamer complexes of the first type and the second type for the different aptamers of the plurality of aptamers.

63. The method of claim 62, comprising identifying respective identification sequences of the different aptamers in the sequence data and generating notifications that the different aptamers are present in the sample based on the identifying.

64. The method of claim 59, wherein generating the sequence data comprises generating sequence reads comprising the identification sequence using a first sequencing primer and generating sequence reads comprising an index associated with the sample using a second sequencing primer.

65. The method of claim 59, wherein generating the sequence data comprises operating a sequencing device to incorporate dark cycles.
